(19) United States
(12) Patent Application Publication
(10) Pub. No.: US 2010/0216693 A1
(43) Pub. Date: Aug. 26, 2010

(54) COMPOSITIONS AND METHODS OF TREATING DIABETES

(76) Inventors: John Wahren, Stockholm (SE); Hans Jornvall, Stockholm (SE); Jawed Shafquat, Stockholm (SE)

Correspondence Address: GLOBAL PATENT GROUP - CBX, 1005 Warson Road, Suite 201, ST. LOUIS, MO, 63132, US

(21) Appl. No.: 11/997,460

(22) PCT Filed: Jul. 27, 2006
(86) PCT No.: PCT/GB06/02821

§ 371 Date: Jun. 1, 2009

Related U.S. Application Data
(60) Provisional application No. 60/704,619, filed on Aug. 1, 2005.

(30) Foreign Application Priority Data
Jan. 31, 2006 (GB) 0601950.9

(51) Int. Cl. A61K 38/28; A61P 3/10; A61P 5/50

(52) U.S. Cl. 514/4

(57) ABSTRACT

The invention features compositions comprising insulin and C-peptide and methods for treating diabetes, using such compositions. The invention further features compositions comprising insulin analogues and C-peptide and the uses thereof for treating diabetes.

### TECHNICAL FIELD

This invention relates to diabetes mellitus and to the formulation and administration of insulin and C-peptide compositions for use in the treatment of diabetes and/or diabetic complications.

### BACKGROUND OF THE INVENTION

Diabetes is generally classified in two main groups. In type 1 diabetes, auto-immune destruction of β-cells within the islets of Langerhans leads to a marked defect in insulin production. In contrast, type 2 diabetes is characterized by insulin resistance in muscle, fat, and liver along with a relative impairment of insulin production in β-cells. Multiple genes contribute to susceptibility in both type 1 and type 2 diabetes, although in most cases their identities remain unknown.

Insulin-dependent diabetes mellitus (IDDM), generally synonymous with type 1 diabetes, is the classical, life-threatening form of diabetes, the treatment of which was revolutionized by the discovery of insulin in 1922. The prevalence of type 1 diabetes is unfortunately widespread throughout much of the world and hence type 1 diabetes represents a serious condition with a significant drain on health resources.

The etiology of type 1 diabetes is multifactorial and not yet entirely clear. However it is characterised by a partial or complete autoimmune destruction of the pancreatic beta cells. In the acute phase of type 1 diabetes insulin deficiency is thus the dominating pathophysiological feature.

After starting insulin treatment many patients enjoy good blood glucose control with only small doses of insulin. There is an early phase, the “honeymoon period”, which may last a few months to a year and which probably reflects a partial recovery of beta cell function. This is, however, a temporary stage and ultimately, the progressive destruction of the beta cells leads to complete cessation of insulin secretion and increasing requirements for exogenous insulin.

While the short term effects of hypoinsulinemia in the acute phase of type 1 diabetes can be well controlled by insulin administration, the long term natural history of type 1 diabetes is darkened by the appearance in many patients of potentially serious complications known as late, or late onset complications. These include the specifically diabetic problems of nephropathy, retinopathy and neuropathy. These conditions are often referred to as microvascular complications even though microvascular alterations are not the only cause. Atherosclerotic disease of the large arteries, particularly the coronary arteries and the arteries of the lower extremities, may also occur.

Nephropathy develops in approximately 35% of type 1 diabetes patients, particularly in male patients and in those with onset of the disease before the age of 15 years. Diabetic nephropathy is characterized by persistent albuminuria secondary to glomerular capillary damage, a progressive reduction of the glomerular filtration rate and eventually, end stage renal failure requiring dialysis treatment or kidney transplantation.

The prevalence of diabetic retinopathy is highest among young-onset type 1 diabetes patients and it increases with the duration of the disease. Proliferative retinopathy is generally present in about 25% of the patients after 15 years duration and in over 50% after 20 years. The earliest lesion of diabetic retinopathy is a thickening of the capillary basement membrane, followed by capillary dilation and leakage and formation of microaneurysms. Subsequently, occlusion of retinal vessels occurs resulting in hypoperfusion of parts of the retina, oedema, bleeding and formation of new vessels as well as progressive loss of vision.

The diabetes-induced nerve disorder is most often a distal symmetric primarily sensory neuropathy affecting 30-50% of type 1 patients. It is often associated with autonomic dysfunction. Sensory neuropathy may cause loss of sensation, appearance of paraaestesia or numbness or, alternatively, result in unpleasant sensations, sometimes pain, in the legs, feet or hands. The morphological changes of diabetic peripheral neuropathy include distal axonal loss with a reduction of the number of large (myelinated) and small fibers, focal demyelinisation and regenerating activity. The function abnormalities include slowing of nerve conduction velocities, reduction of nerve signal amplitudes and rises in sensory modality thresholds. Autonomic neuropathy afflicts approximately 50% of the patients with type 1 diabetes of more than 15 years duration. It may evolve through defects in thermoregulation, impotence and bladder dysfunction followed by cardiovascular reflex abnormalities. Late manifestations may include generalized sweating disorders, postural hypotension, gastrointestinal problems and reduced awareness of hypoglycemia. The latter symptom has grave clinical implications.

A number of theories have been advanced with regard to possible mechanism(s) involved in the pathogenesis of the different diabetic complications but this has not yet been fully elucidated. Metabolic factors may be of importance and it has been shown that good metabolic control is accompanied by significantly reduced incidence of complications of all types. Accordingly, insulin compositions or dosage regimes or therapies which enable or facilitate better glycaemic control by the diabetic patient may be advantageous in this respect in helping to manage and control these complications and/or the risk hereof.

In addition to IDDM (type I diabetes), other types of diabetes are known. Diabetes mellitus is the chronic syndrome of impaired carbohydrate, protein and fat metabolism owing to insufficient secretion of insulin or to target tissue insulin resistance. It occurs in two major forms, type I as discussed above, and type II, non-insulin dependent diabetes mellitus, which differs in etiology, pathology, genetics, age of onset and treatment. Generally, there is no requirement for exogenous insulin in the treatment of type II diabetes. Other forms of diabetes, beyond diabetes mellitus, also exist. Whilst complications, e.g. micro angiopathic complications affecting the retinas and kidneys are seen with higher incidence in type I diabetes, it is not precluded that complications, including those which occur in type I, may occur also with type II diabetes, and other forms of diabetes.

Proinsulin C-peptide is a part of the proinsulin molecule which, in turn, is a precursor to insulin formed in the beta cells of the pancreas. For a long time it was believed that C-peptide (known variously as C-peptide or proinsulin C-peptide) had no role other than as a structural component of proinsulin, facilitating correct folding of the insulin part. However, it has in more recent years been recognised that C-peptide has a physiological role as a hormone in its own right (Wahren et al., (2000), Am. J. Physiol. Endocrinol. Metab, 278, E759-E768). In diabetic patients, it alleviates renal dysfunction, improves blood flow in several tissues, ameliorates nerve functional impairments and is believed to delay or prevent the onset of late complications (Wahren et al., (2000) supra; Johansson J et al. Biochem Biophysical Research Comm. 2002; 295:1035-1040; Wahren et al. in International Textbook of Diabetes, 3rd Edition, editors DeFronzo, Ferrannini, Keen and Zimmet, 2004, Wiley, London). Short term i.v. infusion of C-peptide results in increased whole body glucose utilization, increased skeletal muscle blood flow and oxygen consumption, as well as reduced glomerular hyperfiltration in patients with Type 1 diabetes. Indeed, C-peptide has been proposed for use in the treatment of diabetes in EP 132769 and in SE460334 for use in combination with insulin in the treatment of diabetes and prevention of diabetic complications. Despite these proposals, and increasing evidence for a physiological role for C-peptide, C-peptide therapy, whether alone or in conjunction with insulin, has not yet been widely clinically adopted. In such proposed therapies, C-peptide and insulin are proposed to be administered for the treatment of diabetes on the basis of a therapeutic role each peptide, to achieve and maintain a more natural homeostasis in a diabetic state than may be achieved by administration of insulin alone.

### SUMMARY

The invention is based on the surprising discovery that C-peptide can interact with insulin to reduce insulin aggregation. Thus, the co-administration of insulin and C-peptide prevents insulin aggregation and allows the insulin to act more quickly. This results in increased glucose utilisation, a more pronounced antilipolytic effect and a more marked depression of plasma glucagon levels in Type I diabetic patients compared to administration of insulin alone.

Hence, administration of a combination of insulin, and C-peptide, will produce a more natural utilization of glucose and better glucose control than what is achieved by insulin alone. Accordingly the invention provides compositions, uses of such compositions, and methods of treating diabetes by administering the compositions.

In various aspects the invention provides a composition containing insulin and C-peptide, in particular as defined further below. The insulin may be in the fully processed biologically active form of the hormone or a fragment thereof. By “biologically” active form is meant a fully processed form of insulin capable of promoting, e.g., glucose utilization, carbohydrate, fat and protein metabolism. The insulin may be natural insulin, e.g., human, porcine or bovine. Alternatively, the insulin may be recombinant insulin, chemically synthesized insulin or an insulin analogue. Particular compositions with insulin analogues are discussed further below. The term “C-peptide” as used herein includes all forms of C-peptide (also known as proinsulin C-peptide), including native or synthetic peptides. Such C-peptides are the human peptide, or are from other animal species and genera, preferably mammals. “C-peptide” or “pro-insulin C-peptide” as used herein covers C-peptide isolated from any species.

In certain aspects, in the compositions of the invention the molar ratio of insulin to C-peptide is about 1:1 to 1:5. In such aspects, preferably, the ratio is 1:2. Most preferably, the ratio is 1:4.

In other embodiments, other molar ratios may be used e.g. in the range 1:1 to 1:20 or 10:1 to 1:10. Molar ratios of 10:1 to 5:1 (or 4.2:1) or 1:5 (or 1:4.2) to 1:10 are covered.

Particularly, however, the invention provides compositions in which the molar ratio of insulin to C-peptide is greater than 1:4 (with respect to the C-peptide component) or alternatively put, wherein the ratio of insulin to C-peptide is 1:greater than 4 (1:>4) i.e. 1:4.1, 1:4.2 or 1:4.5 or more e.g. 1:4.1 to 1:10, or 1:4.2 to 1:10, or 1:4.5 to 1:10, more particularly 1:5 to 1:10. Preferred representative molar ratio ranges thus include 1:4.2 to 1:8, 1:4.2 to 1:6, 1:4.5 to 1:8, 1:45 to 1:7, 1:4.5 to 1:6, 1:4.5 to 1:5.5, 1:5-1:8, 1:5-1:7, 1:5 to 1:6. As will be discussed in more detail below, molar ratios in the range of about 1:5 (insulin:C-peptide) (e.g. 1:4.5 to 1:6) have been found to be particularly advantageous and represent a preferred aspect of the present invention.

The pH of the composition is about 5.0 to 8.0, more particular pH 5.0 to 7.0, or pH 5.0 to 6.5, or pH 5.0 to 6.0 with a pH of 5.0 to 5.5 being preferred. Optionally, the composition does not contain zinc. However, again as discussed further below, the C-peptide may particularly have beneficial effects in compositions containing zinc.

The invention also features methods of combating diabetes and/or the complications thereof, e.g. methods of treating or alleviating a symptom of diabetes, e.g., increasing serum insulin levels or decreasing serum glucose levels in a subject, by administering to the subject a composition containing insulin and C-peptide (more particularly a composition of the invention as defined herein). The diabetes may be any diabetes or any diabetic condition. In one embodiment, the diabetes is Type I diabetes. Alternatively, the diabetes is Type 2 diabetes. The subject is a mammal such as human, a primate, dog, cat, or horse. The subject is suffering from diabetes or a diabetic condition. A subject suffering from diabetes is identified by methods known in the art such as determining blood glucose levels. For example, a blood glucose value above 140 mg/dL on at least two occasions after an overnight fast means a person has diabetes. A person not suffering from or at risk of developing diabetes is characterized as having fasting sugar levels between 70-110 mg/dL.

Symptoms of diabetes include fatigue, nausea, frequent urination, excessive thirst, weight loss, blurred vision, frequent infections and slow healing of wounds or sores, blood pressure consistently at or above 140/90, HDL cholesterol less than 35 mg/dL or triglycerides greater than 250 mg/dL, hyperglycemia, hypoglycemia, insulin deficiency or resistance. Diabetic or pre-diabetic patients to which the compounds are administered are identified using diagnostic methods known in the art.

The composition when administered to a subject increases the insulin sensitivity index of the subject. By insulin sensitivity index it is meant the ratio between the area under the concentration curve for plasma glucose and that for plasma insulin after subcutaneous injection of insulin plus C-peptide. By increase it is meant 5%, 10%, 15%, 20%, 25%, 30%, 50%, 75% or greater insulin sensitivity index of the subject compared to a subject that has administered insulin alone.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a graph showing the global fitting (black continuous line) of the experimental recordings (black doted line) in insulin interaction to C-terminally immobilized insulin. The model gives two binding sites in the analyte, binding to one binding site in the immobilized ligand;

FIG. 2A and FIG. 2B are graphs showing the effect of C-peptide (in A, continuous lines) and lack of effect of scrambled C-peptide (in B, continuous lines) on the insulin-insulin interaction. Dashed lines indicate the control measurements in the absence of C-peptide (FIG. 2A) or scrambled C-peptide (FIG. 2B). Insulin concentrations are 1 (bottom curve), 5, 10 and 20 (top curve) μM: C-peptide/scrambled C-peptide concentrations are 5-x. Each line in the graphs is the average of three runs after blank subtraction;

FIG. 3 is a series of nano-ES mass spectra of proinsulin C-peptide, scrambled C-peptide and insulin at 30 μM. Panel A shows dimeric state of insulin (upper section), heterodimer of insulin with C-peptide (middle section) and with scrambled C-peptide (lower section). Panel B shows hexameric charge states of insulin (upper section) and their absence in the presence of 30 μM C-peptide (middle section) or 30 μM scrambled C-peptide (lower section). In section B oligomeric states of C-peptide and scrambled C-peptide can be seen in middle and lower panels respectively;

FIG. 4 is a graph showing the plasma concentration of C-peptide after subcutaneous (s.c) injection of C-peptide (60 nmol) plus soluble insulin (10U;) or insulin and diluent only (∘) into the abdominal wall at time zero. Means±SEM are shown;

FIG. 5 presents a series of nano-ES mass spectra of proinsulin C-peptide, scrambled C-peptide and insulin. Section A shows heterodimers of insulin with C-peptide (lower panel) and their absence with scrambled C-peptide (upper panel) at 3 μM in a ratio of 1:1. Section B shows hexameric charged states of insulin at 30 μM (upper panel) and their absence in presence of 30 μM C-peptide;

FIG. 6 is a graph showing plasma glucose concentrations (left scale) and mean glucose infusion rate during the infusion time (right scale) after s.c injection of C-peptide (60 nmol) plus soluble insulin (10U; and hatched area) or insulin and diluent only (∘ and open area). The injections were performed as in FIG. 4. Means±SEM are shown;

FIG. 7 is a bar diagram showing the area under the concentration curve (AUC) for plasma free fatty acid concentrations 0-180 min and 0-360 min after s.c injection of C-peptide (60 nmol) plus insulin (10U; black bars) or insulin an diluent only (hatched bars). The injections were performed as in FIG. 4. Means±SEM are shown;

FIG. 8 is a bar diagram presenting the area under the glucagon concentration curves (AUC) for 0-180 min and 0-360 min after s.c. injection of C-peptide (60 nmol) plus insulin (10U) (black bars) or insulin (10U) plus diluent (hatched bars). Injections were performed as in FIG. 4. Means±SE are presented;

FIG. 9 is a schematic representation of insulin C-peptide from different species and C-peptide analogues and fragments;

FIGS. 10A and 10B are line charts from surface plasmon resonance measurements showing the effect of zinc concentration (FIG. 10A) and EDTA concentration (FIG. 10B) on insulin-insulin interaction at pH 5 and an insulin concentration of 10 μM;

FIG. 11 is a line chart showing the influence of pH on insulin-insulin interaction in the presence (upper curve) or absence (lower curve) of C-peptide. The concentrations used were 10 μM for insulin and 50 μM for C-peptide. All values are blank-subtracted and were obtained at 150 sec into the dissociation phase;

FIG. 12: Average plasma glucose concentrations in four patients after subcutaneous injection of either a combination of insulin and C-peptide (solid symbols) or insulin alone (open symbols). Injections were carried out as described for FIG. 4;

FIG. 13: Average plasma insulin concentrations in four patients after subcutaneous injection of either a combination of insulin and C-peptide (solid symbols) or insulin only (open symbols). Injections were carried out as described for FIG. 4;

FIG. 14: Total amount of glucose needed to be infused in order to avoid hypoglycemia in four patients (1-4) after subcutaneous injection of either a combination of insulin and C-peptide (solid symbols) or insulin alone (open symbols). Injections were carried out as described for FIG. 4;

FIG. 15: Percent increase in insulin-insulin binding in the presence of 1:1 (grey bars) and 1:5 (black bars) of different peptides and insulin in plasmon resonance experiments. Glu 11-Ala and Glu 27-Ala denote C-peptide analogues in which Glu 11 and Glu 27 have been substituted with Ala. Pentapeptide is the C-terminal segment of C-peptide, ESLQ; and

FIG. 16: Nano-ES mass spectra of a mixture of insulin and C-peptide in 1:1 ratio (both at 30 μM, upper panel) and insulin only (30 μM, lower panel), both at pH 5. The charge states labelled with M, D, T and H in upper case normal font represent identified monomers, dimers, trimers and hexamers of insulin, in upper case italics those identified for C-peptide and in lower case those for a C-peptide/insulin heterodimer. Unlabelled peaks represent variable ion adducts of C-peptide oligomers. The peaks after m/z 2100 are shown at magnification ×20 in both panels. The lower panel illustrates the presence of insulin hexamers at 12⁺, 13⁺, 14⁺ and 15⁺ charge states, while the upper panel demonstrates the absence of insulin hexamers in the insulin/C-peptide mixture.

### DETAILED DESCRIPTION

The invention is based upon the discovery that proinsulin C-peptide interacts with insulin hexamers and reduces insulin aggregation. Specifically, the invention is based on the unexpected discovery that co-administration (e.g. by co-injection) of insulin and C-peptide prevents insulin aggregation and results in increased glucose utilization, a more pronounced antilipolytic effect and a more marked depression of plasma glucagon levels in Type I diabetic patients compared to administration (e.g. injection) of insulin alone.

Insulin is synthesized as pre-proinsulin in the beta cells of the islets of Langerhans. The signal sequence is cleaved off at the entry of proinsulin into the Golgi complex. Inside the Golgi complex, the central part of the molecule undergoes folding, enabling the formation of disulfide bridges between two terminal parts of the molecule. This folded conformation of proinsulin spontaneously dimerizes, and the dimers accumulate around zinc atoms, abundant in this environment, to acquire a stable hexameric complex. In this conformation, the central region of proinsulin is cleaved off by local membrane bound proteases and constitutes the C-peptide. It stays in granules surrounding insulin and is finally secreted into the circulation in a ratio equimolar with insulin. The life of insulin inside granules has been well investigated, and after cleavage of C-peptide, the insulin hexamers get even more tightly associated and sometimes form crystals. The pH in the granular lumen is around 5.5, which further stabilizes the insulin structure. In response to hyper-glycemia, insulin is released along with C-peptide to the circulation where the pH is 7.4. This pH change has been proposed to facilitate the quick solubilization of insulin into the physiologically active monomer from the compact hexameric structure. In the case of diabetic patients, the insulin administered is also hexameric, often with excess of zinc. At the subcutaneous injection site, it takes time for insulin to dissolve and be absorbed. Not all insulin becomes solubilized, and precipitates of insulin have been reported with time at the site of injections. Thus, present modes of administering insulin are not entirely satisfactory, and a continuing need exist to develop new administration forms which may improve insulin action and/or availability.

Patients with Type 1 diabetes mellitus show pronounced destruction of beta cells leading to deficient insulin and C-peptide secretion. As noted above, C-peptide has long been considered to lack biological effects in humans. However, it has recently been demonstrated that C-peptide exerts both metabolic and vascular effects in Type 1 diabetic patients. Short term i.v. infusion of C-peptide results in increased whole body glucose utilization, increased skeletal muscle blood flow and oxygen consumption, as well as reduced glomerular hyperfiltration in patients with Type 1 diabetes. It has been proposed that C-peptide be administered to diabetic patients to be delay or prevent the onset of diabetic complications (Wahren et al., 2000, Supra; Wahren et al., 2004, Supra), or indeed to help manage the treatment of such complications.

Using both surface plasmon resonance (SPR) biosensor instrumentation and electrospray ionization mass spectrometry (ESI-MS) the interactions of monomers of the two peptides lacking in diabetes type 1, insulin and C-peptide, were studied. Interaction between insulin monomers was detected with both methods and influenced by the specific presence of C-peptide. In SPR, the insulin-insulin interaction is noticeable with chip-attached insulin versus a flow of insulin in solution. Binding is similar with C-terminally and N-terminally chip-attached insulin, and noticeable with insulin solutions down to a concentration of 0.05 μM at pH 5. Binding is pH-dependent with a maximum at pH 5 and is also influenced by zinc ions or zinc chelators. Using ESI-MS, a clear polymerization of insulin is noticeable, with dimerization detectable already at 3 μM insulin, and higher polymers up to hexamers detectable at 30 μM. C-peptide was found to influence all of the insulin-insulin interactions observed. Controls with scrambled C-peptide (the same amino acid composition as native C-peptide but with the residues assembled in a random order) showed a considerably weaker effect compared to C-peptide in either method, demonstrating C-peptide specificity for the insulin interaction. In ESI-MS, C-peptide was found to significantly lower the insulin polymerization noticeable at both 1-fold and 5-fold C-peptide excess over insulin at the μM level. In SPR, C-peptide was found to increase observable binding to chip-bound insulin when mixed into a flow of insulin solution, but not to give any observable binding when alone in solution. The binding promoting effect of C-peptide was most clearly observed at higher insulin concentrations (>0.5 μM). These results indicate that C-peptide promotes chip-binding of insulin by depolymerization of insulin at high concentration. Thus, C-peptide may help to “reduce” or “prevent” insulin aggregation, essentially, it is believed, by causing disaggregation of any aggregates which may form. Physiologically, these effects are relevant in the secretory granules in the pancreas islets of healthy individuals, and at the injection sites of diabetic subjects.

Specifically, at such sites, insulin is present at high concentration and C-peptide, when administered, or delivered, in combination with the insulin, may act to promote disaggregation of any insulin complexes which may have formed. In this way C-peptide may act to promote availability of the insulin, and allow it to act more quickly. C-peptide may accordingly act to improve or promote insulin action.

Further, as noted above, insulin is conventionally administered formulated with zinc. In such formulations the presence of zinc will promote insulin aggregation, and the insulin will generally be present in hexameric complexed form. By including C-peptide in such insulin formulations, disaggregation of the insulin complexes may be promoted, thus ensuring that the insulin is present in the composition in a more bio-available, or rapid-acting form.

The results of these studies demonstrate that C-peptide if mixed with insulin at injection may promote insulin availability and efficiency in diabetic subjects. To test this hypothesis the interaction of C-peptide and insulin with regard to subcutaneous absorption kinetics, glucose utilization and lipolysis was evaluated. Nine Type 1 diabetic patients (4 female, 5 male) with a mean age of 322 yrs and a diabetes duration of 17±2 yrs were studied on two separate occasions. Insulin was infused i.v. during the night prior to each experiment. Biosynthetic human C-peptide (60 nmol) plus an equimolar amount of soluble human insulin (10 U; CI-day) or C-peptide diluent and soluble insulin (I-day) were injected subcutaneously into the abdominal wall in a double blind randomized study design. Plasma levels of C-peptide, free insulin, glucose, free fatty acids (FFA) and glucagon were determined intermittently for six hours. In order to avoid hypoglycemia a variable glucose infusion was started at a plasma concentration of 3.5 mmol/l. On the CI-day peak plasma C-peptide levels of 2.1±0.2 nmol/l were found after 57±3 min. Plasma C-peptide concentration above the physiological level (>0.3 nmol/l) was maintained for 4 to 5 hours. Maximal plasma insulin levels of 27±4 and 28±3 μU/ml were found at 88±13 and 88±11 min on the CI- and I-days, respectively (n.s.). The amounts of glucose needed to avoid hypoglycemia were 66% (p<0.02) greater on the CI-day than on the I-day. The AUC for FFA (0-180 min) concentrations was smaller on the CI-day than on the I-day (p<0.05). Likewise, the AUC for glucagon concentrations (0-360 min) was smaller after the combined injection than after insulin alone (p<0.05).

These results indicate that subcutaneous injection of an ordinary meal dose of insulin, in combination with an equimolar dose of biosynthetic human C-peptide, resulted in maintenance of physiological plasma C-peptide levels during 4-5 hours, without changing the absorption kinetics of the s.c. injected insulin. Furthermore, compared to insulin alone, the combination of C-peptide and insulin caused a more pronounced increase in glucose utilization, a greater depression of lipolysis and a more pronounced reduction of plasma glucagon concentration.

Further our work has also shown that at higher molar ratios of C-peptide to insulin (e.g. insulin C-peptide ratios of 1:5), a particularly beneficial disaggregating effect of C-peptide on insulin may be observed. Such insulin:C-peptide ratios (e.g. in the region of 1:5 insulin:C-peptide) may also be clinically favourable or advantageous in terms of administering C-peptide to the patient (which as noted above has beneficial physiological effects in its own right).

The results presented herein (see further in the Examples below) accordingly show a heretofore unexpected, and surprising, beneficial effect of C-peptide in preventing or reducing insulin aggregation, and in promoting insulin action (or effect).

In one aspect, the present invention accordingly provides the use of C-peptide in reducing aggregation of insulin.

Thus, C-peptide may be added to, or included in, compositions containing insulin.

C-peptide may accordingly be used to prepare or produce pharmaceutical compositions containing insulin. Such compositions may be administered to patients and the C-peptide contained therein may have a beneficial effect in reducing aggregation of the insulin. As noted above, it is believed that this results from disaggregation of the insulin. Thus the C-peptide may promote or assist in the disaggregation of insulin or cause its disaggregation (i.e. C-peptide may act to disaggregate insulin, whether insulin hexamers formed in the presence of zinc or any other aggregated insulin forms e.g. insulin dimers etc.).

C-peptide when administered in combination with insulin (i.e. delivered to the same administration site) may act to reduce aggregation of any insulin administered in aggregated form (e.g. in hexameric form complexed with zinc) or may reduce any aggregation of insulin which occurs upon administration.

Accordingly, in a related aspect the present invention also provides the use of C-peptide in the manufacture of an insulin-containing preparation for reducing the aggregation of insulin in said preparation.

By reducing the aggregation of insulin in the preparation, the insulin effect of the preparation may be improved or augmented, e.g. the insulin may become more rapid-acting (the rate of insulin action may be increased).

Alternatively viewed, such aspects of the present invention also provide a method of reducing insulin aggregation in vivo, comprising administering to a subject in need thereof a composition (or combination) comprising insulin and C-peptide.

Also provided is a method of reducing insulin aggregation comprising adding C-peptide to, or including C-peptide in, a composition containing insulin.

It will be clear from the above, that as used herein the term “reducing aggregation of insulin” includes all forms of reducing insulin aggregation. Thus the degree or amount of aggregation observed (e.g. in the composition or at the administration site) may be reduced as compared to in the absence of the C-peptide (i.e. insulin alone). Insulin aggregation may be prevented. Thus, “reducing” or “reduced” “aggregation” includes no observable aggregation or reduced amounts of aggregation (i.e. reduced levels of aggregated insulin). Thus, for example the amount of insulin aggregates present in the composition, or at the administration site may be reduced by at least 10, 20, 30, 40, 50, 60, 70, 80, 90 or 100% as compared to the amount of aggregates in the absence of C-peptide. Insulin aggregation may be reduced by disaggregation of any insulin aggregates which are present or which have formed. C-peptide may thus cause, promote or assist in the disaggregation of insulin aggregates.

The terms “aggregated insulin” or “insulin aggregates” as used herein include any form of insulin aggregate i.e. any non-monomeric form. Thus dimers and any higher oligomers are included, including in particular hexameric insulin complexes. Such aggregated forms may be formed in the presence or absence of zinc.

Thus, C-peptide, when in contact with aggregated insulin (e.g. hexameric insulin as provided in commercial insulin preparations) facilitates its disaggregation into dimers, and eventually monomers, the biologically active form.

Whilst not wishing to be bound by the theory, it is believed that the C-peptide causes the insulin to be absorbed not only more rapidly from the administration site (e.g. subcutaneous space), but also more reproducibly and completely. This thus helps to address a clinical problem often encountered in clinical practice, namely that the effect of an insulin injection varies considerably from day to day, and between patients.

Therapeutic Compositions

The invention provides compositions containing C-peptide and insulin. In certain embodiments, insulin is present in a molar ratio, insulin to C-peptide, of from about 1:1 to about 1:5. Preferably, the molar ratio of insulin to C-peptide is about 1:2. As discussed further below, other molar ratio ranges are also advantageous and are encompassed by the invention.

The compositions of the invention comprise the active ingredients, insulin, and C-peptide together with a pharmaceutically acceptable carrier and, optionally, other therapeutic ingredients. The carrier must be acceptable in the sense that it is compatible with other components of the composition and is not deleterious to the recipient. The composition may not contain zinc.

An acid, such as hydrochloric acid, or a base, such as sodium hydroxide, can be used for pH adjustment. In general, the pH of the aqueous composition ranges from about 2 to about 7, and, preferably, from about 5.0 to about 5.5.

As noted above, broadly speaking, molar ratios in the ranges 1:1 to 1:20 or 10:1 to 1:10 insulin:C-peptide are encompassed. The invention accordingly provides compositions (or combinations) in which the molar ratio ranges of 10:1 to 5:1 (or 10:1 to 4.2:1) or 1:5 to 1:10 (or 1:4.2 to 1:10) are covered.

In particular it has been found that compositions or combinations in which C-peptide is present in a molar excess of greater than 4-fold over insulin are particularly advantageous, both from the point of view of the disaggregating effect of C-peptide on insulin aggregates and also clinically in terms of C-peptide delivery to the subject (i.e. patient).

Thus, a composition comprising insulin and C-peptide together with at least one pharmaceutically acceptable excipient or carrier wherein said C-peptide is present at a molar excess of greater than 4-fold with respect to said insulin, represents a preferred aspect of the invention.

More particularly the C-peptide is present in at least 4.2-fold, at least 4.5-fold or at least 5-fold molar excess over insulin (i.e. wherein the molar ratio of insulin:C-peptide is greater than 1:4 with respect to the C-peptide, e.g. insulin to C-peptide ratios of at least 1:4.2, 1:4.5 or 1:5, with respect to the C-peptide, i.e. insulin to C-peptide ratios of 1:4.2, 1:4.5, 1:5 or more, e.g. 1:4.2-1:10, 1:4.5-1:10, 1:5-1:10.

As referred to herein, an insulin:C-peptide molar ratio of greater than 1:4 means a molar ratio of insulin to C-peptide in which the level of C-peptide relative to insulin is greater than 4:1 (e.g. 4.2:1 or more, or 4.5:1 or more).

Preferred representative molar ratio ranges thus include 1:4.2 to 1:8, 1:4.2 to 1:6, 1:4.5-1.8, 1:4.5-1:7, 1:4.5-1:6, 1:4.5-1:5.5, 1:5-1:8, 1:5-1:7, 1:5-1:6.

Exemplary molar ratios thus include 1:4.2, 1:4.5, 1:5, 1:5.5, 1:6, 1:6.5 and 1:7.

Alternatively put, the C-peptide is present in a molar amount of greater than 80% relative than insulin (i.e. the molar amount of insulin is less than 20%), more particularly at least 81%, 82%, 83%, 84% or 85% C-peptide is present on a molar basis, with respect to insulin.

The pH of the compositions of the invention may lie in the range of about 5.0 to 8.0, more particularly, pH 5.0 to 7.5, 5.0 to 7.0, 5.0-6.9, 5.0-6.5, 5.0-6.1, 5.0-6.0 or 5.0 to 5.5. Hence, the pH of the composition may be 6.1, 6.2, 6.3, 6.4, 6.5, 6.6, 6.7, 6.8 or 6.9.

Experiments with the effect of C-peptide on insulin binding in the laboratory SPR biosensor studies have suggested that the effect of C-peptide on insulin interaction is most pronounced at pH 5-6. However, in the clinical studies C-peptide was used in conjunction with a commercial insulin preparation at physiological pH and an effect of the C-peptide was observed on insulin effect. Accordingly, these studies support that C-peptide may exert its effects on insulin interaction (i.e. aggregation) at around physiological pH, and hence compositions at around physiological pH i.e. in the range pH 7.0 to pH 7.5 are covered. Thus ranges of pH 5.0 to 7.5, 5.0 to 7.4, 5.0 to 7.3 or 5.0 to 7.2, 5.0 to 7.1 and 5.0 to 7.0 are included.

Compositions of the invention suitable for administration may comprise sterile aqueous solutions and/or suspensions of the pharmaceutically active ingredients, which solutions or suspensions preferably are made isotonic, e.g. with sodium chloride, glycerin, glucose, mannitol, or sorbitol. In addition, the compositions may contain any of a number of adjuvants, such as buffers, preservatives, dispersing agents, agents that promote rapid onset of action, and agents that promote prolonged duration of action. Typical preservatives are, for example, phenol, m-cresol, and methyl p-hydroxybenzoate. Typical buffers are, for example, sodium phosphate, sodium acetate, and sodium citrate.

As noted above, zinc may promote aggregation of insulin into hexameric form. Accordingly, in certain embodiments it may be thought desirable to avoid zinc in the compositions and preparations according to the invention and compositions which do not contain zinc are expressly encompassed.

On the other hand, commercial insulin preparations often contain zinc (the insulin is present in zinc-complexed form). C-peptide may be used according to the present invention in conjunction with such compositions, and may exert a beneficial effect in promoting disaggregation of the insulin. Accordingly, in other embodiments, zinc-containing compositions are included, particularly where the insulin is supplied in zinc-complexed form.

Insulin is available via a variety of routes, including organic synthesis, isolation from human pancreas by conventional methodology, and recombinant DNA methodology. The term “insulin” as used herein is meant to include insulin and active fragments and analogues thereof. In one embodiment, the insulin is human insulin, or insulin from any other species. Alternatively, the insulin is non-human insulin such as porcine or bovine insulin. Many types of insulin are known in the art. The insulin is very fast acting, fast acting, intermediate acting, long acting or ultra-long-acting. Exemplary nucleic acids and polypeptides encoding insulin include for example human insulin are known in the art. Additional sources include commercially available insulin such as Novolin R®, Humulin R®, Reg/Iletin II Pork®, Velosulin®, Lente, Ultralente, NPH, Isophane, NPH/Iletin II Pork®, Novolin N®, Humulin N®, Humulin L®, Humulin U or Novolin L®. Insulin analogues include for example, Lispro, Aspart and Glargine. Other sources of insulin are described in Hirsch, N Engl J Med 2005; 352:174-83, which is hereby incorporated by reference in its entirety.

Thus any insulin is included, and the term “insulin” includes all forms of insulin, including native and synthetic peptides, fragments of insulin molecules and sequence-modified variants of insulin (e.g. insulin analogues). The insulin may thus be a native insulin (e.g. isolated), recombinant insulin, chemically-synthesised insulin or an insulin analogue.

The insulin may be human insulin or may be from other animal species and genera, preferably mammal. Preferably, the insulin is human insulin having the amino acid sequence of SEQ ID NO. 28 (FVNQHLCGSHLVEALYLVCGERGFFYTPKT) for insulin A chain and SEQ ID NO. 29 (GIVEQCCTSICSLYQLENYCN) for insulin B chain which are derivable from SEQ ID NO. 30 (MALWMRLLPLLALLALWGPDPAAAFVNQHLCGSHL VEALYLVCGERGFFYTPKTRREAEDLQVGQVELGGGPGAGSLQPLALEGSL QKRGIVEQCCTSICSLYQLENYCN) the insulin precursor.

Variants of human insulin are included which may be native variants or synthetically or artificially derived. Insulins from a number of different species have been sequenced and are known in the art. It would thus be a routine matter to select a variant being an insulin from a species or genus other than human. Thus variants and modifications of native insulin are included as long as they retain insulin activity. Insulin may be in its native form i.e. as different variants as they appear in nature in different species which may be viewed as functionally equivalent variants of human insulin or they may be functionally equivalent derivatives thereof, which may differ in their amino acid sequence for example by truncation (e.g. from the N- or C-terminus or both) or other amino acid deletions, additions, insertions or substitutions. It is known in the art to modify the sequences of proteins or peptides while retaining their useful activity and this may be achieved using techniques which are standard in the art and widely described in the literature e.g. random or site directed mutagenesis, cleavage and ligation of nucleic acids. For example, a sequence modified variant of insulin (e.g. an insulin analogue) may act as insulin in decreasing blood glucose levels.

Any such modifications or combinations thereof may be made as long as insulin activity is retained. Thus insulin which may be used according to the invention may have an amino acid sequence (or more particularly A and B chain amino acid sequences), which is (are) substantially homologous or substantially similar to the native insulin amino acid sequences, for example to human insulin A chain of SEQ ID NO. 28 and B chain of SEQ ID NO. 29 Thus, insulin may have an amino acid sequence having at least 30%, preferably at least 40, 50, 60, 70, 75, 80, 85, 90, 95, 98 or 99% identity to that of human insulin of SEQ ID NO. 28 or 29.

Amino acid sequence identity or similarity may be determined for example using the BestFit program of the Genetics Computer Group (GCG) from the University of Wisconsin. The program may use the local homology algorithm of Smith and Waterman with the default values: Gap creation penalty=8, Gap extension penalty=2, Average match=2.912, Average mismatch=2.003.

Functionally equivalent derivatives or variants of native insulin sequences may readily be prepared according to techniques well known in the art, and include peptide sequences having a functional, e.g. a biological, activity of a native insulin.

Fragments of native or synthetic insulin sequences may also have the desirable functional properties of the insulin from which they are derived. The term “fragment” as used herein includes fragments of insulin provided that the fragment retains the biological or therapeutically beneficial activity of the whole molecule.

The term “derivative” or “analogue” as used herein thus refers to insulin sequences or fragments thereof, which have modifications as compared to the native sequence. Such modifications may be one or more amino acid deletions, additions, insertions and/or substitutions. These may be contiguous or non-contiguous.

Representative variants may include those having 1 to 6, or more preferably 1 to 4, 1 to 3 or 1 or 2 amino acid substitutions, deletions and/or insertions as compared to SEQ ID NO. X or X₁. The substituted amino acid may be any amino acid, particularly one of the well known 20 conventional amino acids (Ala (A); Cys (C); Asp (D); Glu (E); Phe (F); Gly (G); His (H); Ile (I); Lys (K); Leu (L); Met (M); Asn (N); Pro (P); Gln (Q); Arg (R); Ser (S); Thr (T); Val (V); Trp (W); and Tyr (Y)).

Chemical modification of the peptide structure is not precluded e.g. by glycosylation as long as the structure of the derivative or analogue remains essentially peptide in nature. As mentioned above, modification of an amino acid sequence may be by amino acid substitution, for example an amino acid may be replaced by another that preserves the physicochemical character of the peptide (e.g. A may be replaced by G or vice versa, V by A or L; E by D or vice versa; and Q by N). Generally, the substituting amino acid has similar properties e.g. hydrophobicity, hydrophilicity, electronegativity, bulky side chains etc. to the amino acid being replaced. Isomers of the ‘native’ L-amino acid, e.g. D-amino acids may be incorporated.

Additional variants may include amino and/or carboxyl terminal fusions as well as intrasequence insertions of single or multiple amino acids. Longer peptides may comprise multiple copies of one or more of the peptide sequences. C and N-terminal protecting groups may be included. Insertional amino acid sequence variants are those—in which one or more amino acid residues are introduced at a site in the protein. Deletional variants are characterised by the removal of one or more amino acids from the sequence. “Variants” may include, for example, different allelic variants as they appear in nature, e.g. in other species or due to geographical variation. All such variants, derivatives, analogues or fragments of insulin are included, and are subsumed under the general term “insulin”.

The variants, derivatives, analogues and fragments are functionally equivalent in that they retain insulin activity. More particularly, they exhibit at least 40%, preferably at least 60%, more preferably at least 80% of the activity of insulin, particularly human insulin. Thus they are capable of functioning as insulin i.e. can substitute for insulin itself. Such activity means any activity exhibited by a native insulin, whether a physiological response exhibited in an in vivo or in vitro test system, or any biological activity or reaction mediated by a native insulin, for example in an enzyme assay or in binding to test tissues or membranes. Tests for insulin activity may include measuring glucose transport in isolated cells or tissues, for example glucose transport into muscle strips or myoblasts. Alternatively a binding assay may be used to measure binding to known insulin receptors.

The insulin may be provided in different forms which may influence whether it is long-acting, or rapid acting etc. Thus, for example the insulin may be provided in complexed form, for example with zinc or other materials e.g. protamine (e.g. as hexameric zinc complexes). Ultralente insulin for example contains zinc to promote hexamer formation. This slows down the insulin action. Alternatively, the actual insulin itself may be “modified” e.g. sequence modified to form an analogue or derivative which affects its duration of action. Glargine and insulin Determir are other examples of long acting analogues. Lispro and Aspart are examples of short acting analogues which contain small amounts of zinc for improved stability.

Analogues of insulin are of particular interest, particularly where a native insulin has been sequence-modified. This may alter the properties or characteristics of the insulin, for example to alter its duration of action. Various such analogues, in particular sequence-modified analogues of insulin are known and described in the literature and used clinically (see the review by Hirsch, supra). Insulin analogues also encompass native insulin where one or more disulphide bonds are deleted, added or moved and native insulin as a different salt e.g. replacing zinc cations with sodium.

Thus for example in the analogue Lispro (also known as Humalog) the lysine of B29 and the proline of B28 of human insulin are inverted. This confers a conformational change that results in a shift in the normal binding of the C-terminal portion of the B chain which in turn reduces the formation of dimers and hexamers. Such an analogue is therefore “rapid acting” as compared to regular human insulin, and indeed human insulin preparations such as zinc insulin (Lente) which have extended duration: 5-15 minutes onset of action, 30-90 minutes peak action and 4-6 hours of effective duration (as compared to 30-60 minutes onset of action, 2-3 hours of peak action and 8-10 hours effective duration for regular insulin, and 2-4 hours onset of action, 4-10 hours peak action and 12-20 hours effective duration for zinc insulin (Lente).

A second rapidly acting analogue that is commercially available is Aspart (also known as Novo Rapid). Here proline B28 in human insulin has been replaced by aspartic acid. This has a similar action profile to Lispro.

Insulin Glulisine (also known as Apidra) is a further rapidly acting analogue with a pharmacokinetic profile similar to Lispro and Aspart. In this analogue lysine B3 in human insulin is replaced by asparagine and lysine. B29 is replaced by glutamic acid.

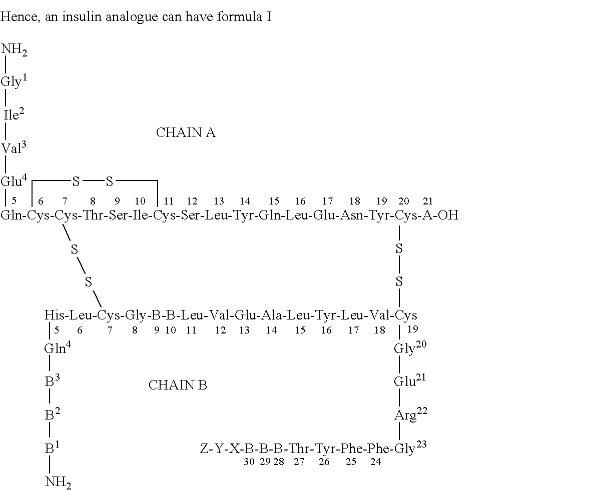

wherein A21 is alanine, asparagine, aspartic acid, glutamine, glutamic acid, glycine, threonine or serine; B1 is phenylalanine, aspartic acid, or is absent; B2 is valine or may be absent when B1 is absent; B3 is asparagine or aspartic acid; B9 is serine or aspartic acid; B10 is histidine or aspartic acid; B28 is any amino acid, B29 is L-proline, D-proline, D-hydroxyproline, or L-hydroxyproline; B30 is alanine, threonine or is absent; Z is —OH, —NH₂, —OCH₃, or —OCH₂CH₃; X is Arg, Arg-Arg, Lys, Lys-Lys, Arg-Lys, Lys-Arg, or is absent; and Y may be present only when X is present and, if present, is Glu or an amino acid sequence which comprises all or a portion of the sequence -Glu-Ala-Glu-Asp-Leu-Gln-Val-Gly-Gln-Val-Glu-Leu-Gly-Gly-Gly-Pro-Gly-Ala-Gly-Ser-Leu-Gln-Pro-Leu-Ala-Leu-Glu-Gly-Ser-Leu-Gln-Lys-Arg- and which begins at the N-terminus Glu of such sequence. Such insulin analogues are described in U.S. Pat. No. 5,514,646 and U.S. Pat. No. 5,700,662 (which are incorporated herein by reference).

In a particular aspect, the present invention provides a composition comprising an insulin analogue and C-peptide, together with at least one pharmaceutically acceptable excipient, wherein said insulin analogue is not a long-acting analogue.

More particularly said insulin analogue is a rapid acting or intermediate-acting analogue (i.e. a rapid to intermediate acting analogue) or has an action profile similar to regular human insulin. “Rapid-acting” is defined herein as effective duration of 4 to 6 hours. Such a rapid-acting analogue may have an onset of action of 5-15 minutes and peak action of 30-90 minutes.

“Long-acting” is defined herein as an effective duration of 20 to 36 hour or 20 to 24 hours. A long acting analogue may have an onset of 2 to 4 hours (or longer) and exhibit no peak action.

Accordingly, an analogue according to this aspect of the invention may have a duration of action of 2 to 18, e.g. 4 to 18 hours or 2 to 16 e.g. 4 to 16 hours, more particularly 2-15 hours, 4-15 hours, 4-12 hours, 2-10 hours, 4-10 hours, or 2-8, 2-6, 2-5 hours or 3-10, 3-8, 3-6 or 3-5 hours. Onset of action may be from 5 minutes to 4 hours, e.g. 5 minutes to 3 hours or 5 minutes to 2 hours; 5 to 90 or 60 minutes or 5 to 30 minutes or 20 to 40 minutes. Peak action may be at 0.5 to 12 hours e.g. 0.5 to 10 hours.

Thus an analogue of this aspect of the invention may be a sequence-modified variant of human insulin (SEQ ID NO. 28 and 29) as defined above, (e.g. having an amino acid sequence having at least 30% (or higher, as defined above) identity to SEQ ID NO. 28 and 29, wherein said analogue has a duration of action of 2 to 18 hours.

Preferred analogues according to this aspect of the invention include Aspart, Lispro and Glulisine.

Rapid-acting analogues such as Lispro are designed to reduce dimerisation and hence it might be expected that they exhibit reduced aggregation and hence C-peptide may have a more limited effect. Nonetheless, in the clinical environment, aggregation of such analogues may still occur and C-peptide may have a beneficial effect. Furthermore, such analogues may be formulated with zinc, which would tend to promote aggregation, and in such circumstances the presence of C-peptide may have a beneficial disaggregating effect.

Various long-acting analogues of insulin have been described which have been modified to introduce a basic organic group at the C-terminal of the B chain. Such a basic group may be Arg-OH or Arg-Arg-OH and glargine is such a commercially available analogue carrying an additional C-terminal Arg-Arg on the B chain (at B30) and a Gly substitution for asparagine at A21. Such long-acting analogues are described in U.S. Pat. No. 4,608,364 and are expressly excluded from this aspect of the invention (i.e. are excluded as being long-acting analogues).

In a particular embodiment of this aspect, therefore the insulin analogue is not an insulin derivative of Formula I of U.S. Pat. No. 4,608,364. Thus, the insulin analogue is not an insulin derivative of the formula I

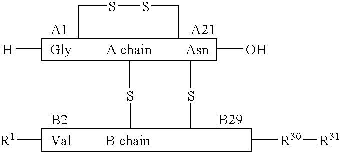

in which

R¹ denotes H or H-Phe,

R³⁰ represents the radical of a neutral L-amino-acid which can be genetically coded and

R³¹ represents a physiologically acceptable organic group of basic character with up to 50 carbon atoms, in the build-up of which 0 to 3 α-amino-acids participate and in which the terminal carboxyl function optionally present can be free, as an ester function, as an amide function, as a lactone or reduced to CH₂OH.

In this aspect of the invention where the insulin is a non-long-acting insulin analogue, it is preferred that the composition does not also contain a native unmodified insulin, e.g. human insulin. More particularly, the insulin analogue is the sole “insulin” component.

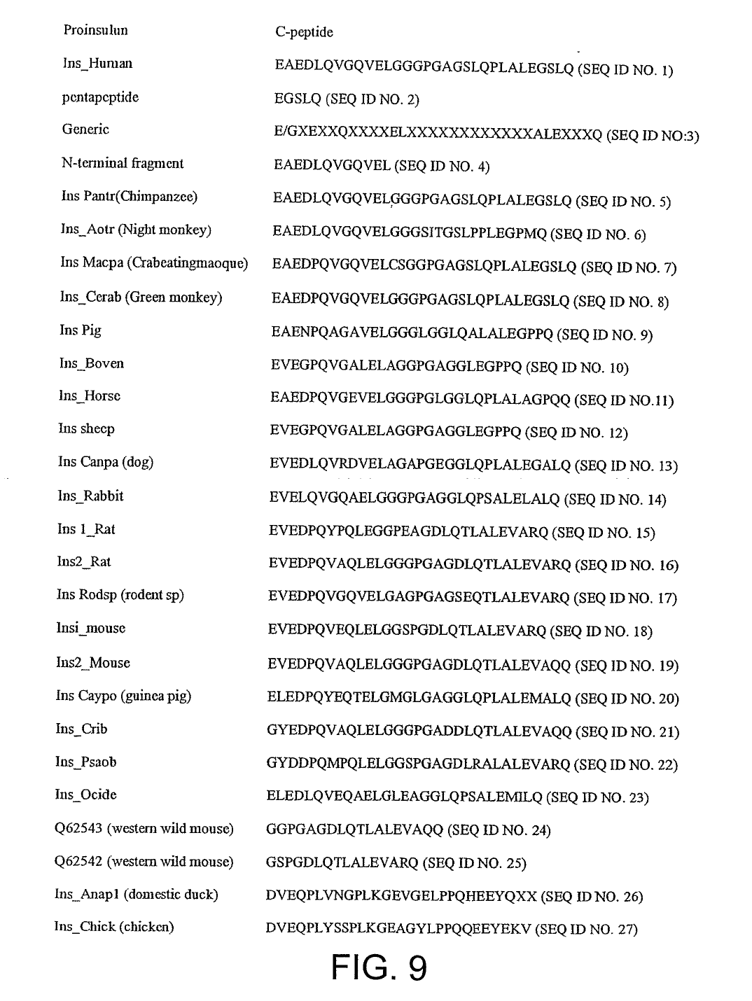

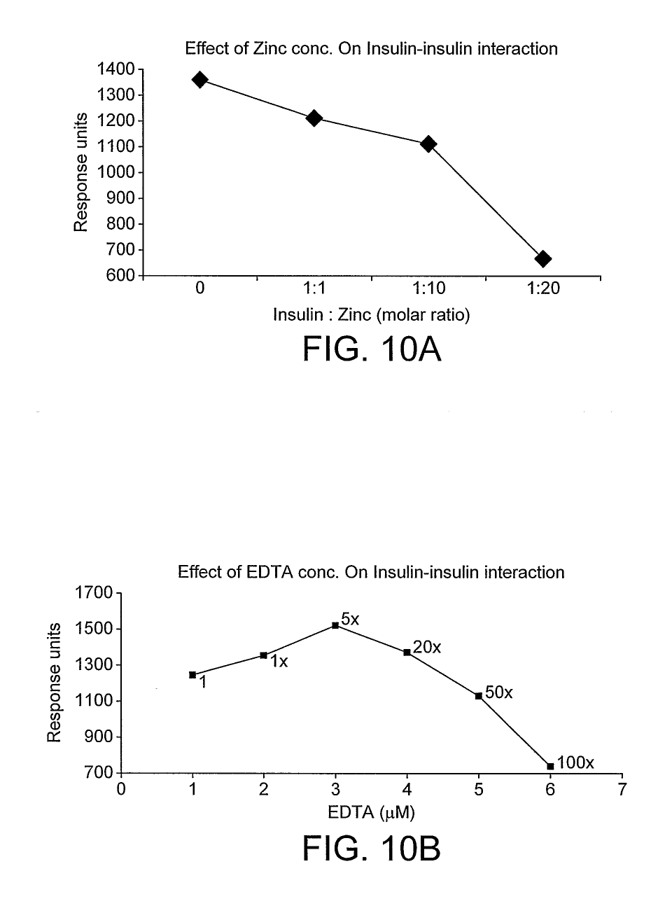

C-peptide is a part of the proinsulin molecule that is a precursor to insulin formed in the beta cells of the pancreas. The term “C-peptide” as used herein includes all forms of C-peptide (also known as proinsulin C-peptide), including native or synthetic peptides. Such C-peptides are the human peptide, or are from other animal species and genera, preferably mammals. “C-peptide” or “pro-insulin C-peptide” as used herein covers C-peptide isolated from any species. Preferably, “C-peptide” refers to human C-peptide having the amino acid sequence EAEDLQVGQVELGGGPGAGSLQPLALEGSLQ (SEQ ID NO. 1). Variants of human C-peptide are included, which may be native variants, or synthetically or artificially derived. C-peptides from a number of different species have been sequenced and are known in the art. It would thus be a routine matter to select a variant being a C-peptide from a species or genus other than human. Several such variants of C-peptide (i.e. representative C-peptides from other species) are shown in FIG. 9 (see SEQ ID NOS. 1 and 5-27). Thus variants and modifications of native human C-peptide are included as long as they retain C-peptide activity. The C-peptides may be in their native form, i.e., as different variants as they appear in nature in different species which may be viewed as functionally equivalent variants of human C-peptide or they may be functionally equivalent derivatives thereof, which may differ in their amino acid sequence, for example by truncation (e.g. from the N- or C-terminus or both) or other amino acid deletions, additions, insertions or substitutions. It is known in the art to modify the sequences of proteins or peptides, while retaining their useful activity and this may be achieved using techniques which are standard in the art and widely described in the literature, e.g., random or site-directed mutagenesis, cleavage and ligation of nucleic acids.

Any such modifications, or combinations thereof, may be made, as long as activity is retained. The C-terminal end of the molecule known to be important for activity. Preferably, therefore, the C-terminal end of the C-peptide should be preserved in any such C-peptide variants or derivatives, more preferably the C-terminal pentapeptide of C-peptide should be preserved or sufficient. (See, Henriksson, M., et al, 2005, Cell Mol. Life Sci.; 62, 1772-1778).

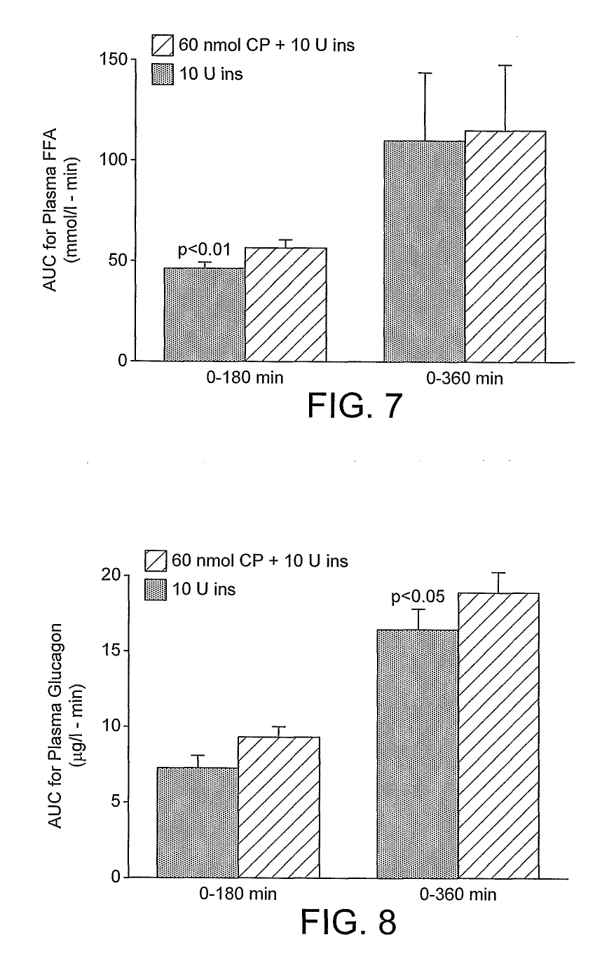

Modifications to the mid-part of the C-peptide sequence (e.g. to residues 13 to 25 of human C-peptide) allow the production of functional derivatives or variants of C-peptide. Thus, C-peptides which may be used according to the invention may have amino acid sequences which are substantially homologous, or substantially similar to the native C-peptide amino acid sequences, for example to the human C-peptide sequence of SEQ ID NO. 1 or any of the other native C-peptide sequences shown in FIG. 8. Alternatively, the C-peptide may have an amino acid sequence having at least 30% preferably at least 40, 50, 60, 70, 75, 80, 85, 90, 95, 98 or 99%) identity with the amino acid sequence of any one of SEQ ID Nos. 1 or 5-27 as shown in FIG. 9, preferably with the native human sequence of SEQ ID No. 1. Although any amino acid of C-peptide may be altered as described above, it is preferred that one or more of the glutamic acid residues at positions 3, 11 and 27 of human C-peptide (SEQ ID NO. 1) or corresponding or equivalent positions in C-peptide of other species, are conserved. Preferably, all of the glutamic acid residues at positions 3, 11 and 27 (or corresponding Glu residues) of SEQ ID NO. 1 are conserved. Alternatively, it is preferred that Glu27 (or a corresponding Glu residue) is conserved. An exemplary functional equivalent includes the amino acid sequence: E/GXEXXQXXXXELXXXXXXXXXXXXALEXXXQ (SEQ ID NO:3). As used herein, X is any amino acid. The single residue represented by E/G may be either Glu or Gly in SEQ ID NO.3.

Amino acid sequence identity or similarity may be determined for example using the BestFit program of the Genetics Computer Group (GCG) from the University of Wisconsin. The program may use the local homology algorithm of Smith and Waterman with the default values: Gap creation penalty=8, Gap extension penalty=2, Average match=2.912, Average mismatch=2.003. Functionally equivalent derivatives or variants of native C-peptide sequences may readily be prepared according to techniques well known in the art, and include peptide sequences having a functional, e.g. a biological, activity of a native C-peptide.

Fragments of native or synthetic C-peptide sequences may also have the desirable functional properties of the peptide from which they derived. The term “fragment” as used herein thus includes fragments of a C-peptide provided that the fragment retains the biological or therapeutically beneficial activity of the whole molecule. The fragment may also include an C-terminal fragment of C-peptide. Preferred fragments comprise residues 15-31 of native C-peptide, more especially residues 20-31. Peptides comprising the pentapeptide EGSLQ (SEQ ID NO. 2) (residues 27-31 of native human C-peptide) are also preferred. The fragment may thus vary in size from e.g., 4 to 30 amino acids or 5 to 20 residues. Suitable fragments are disclosed in WO 98/13384 the contents of which are incorporated herein by reference. The fragment may also include an N-terminal fragment of C-peptide, typically having the sequence EAEDLQVGQVEL (SEQ ID NO. 4), or a fragment thereof which comprises 2 acidic amino acid residues, capable of adopting a conformation where said two acidic amino acid residues are spatially separated by a distance of 9-14 Å between the alpha-carbons thereof. Also included are fragments having N and/or C-terminal extensions or flanking sequences. The length of such extended peptides may vary, but typically are not more than 50, 30, 25 or 20 amino acids in length. Our suitable fragments are described in U.S. Pat. No. 6,610,649, which is hereby incorporated by reference in its entirety.

In such a case it will be appreciated that the extension or flanking sequence will be a sequence of amino acids which is not native to a naturally-occurring or native C-peptide, and in particular a C-peptide from which the fragment is derived. Such a N- and/or C-terminal extension or flanking sequence may comprise e.g., from 1 to 10, e.g., 1 to 6, 1 to 5, 1 to 4 or 1 to 3 amino acids.

The term “derivative” as used herein thus refers to C-peptide sequences or fragments thereof, which have modifications as compared to the native sequence. Such modifications may be one or more amino acid deletions, additions, insertions and/or substitutions. These may be contiguous or non-contiguous.

Representative variants may include those having 1 to 6, or more preferably 1 to 4, 1 to 3 or 1 or 2 amino acid substitutions, insertions and/or deletions as compared to SEQ ID No. 1. The substituted amino acid may be any amino acid, particularly one of the well known 20 conventional amino acids (Ala (A); Cys (C); Asp (D); Glu (E); Phe (F); Gly (G); His (H); Ile (I); Lys (K); Leu (L); Met (M); Asn (N); Pro (P); Gln (Q); Arg (R); Ser (S); Thr (T); Val (V); Trp (W); and Tyr (Y)).

Chemical modification of the peptide structure is not precluded e.g. by glycosylation as long as the structure of the derivative remains essentially peptide in nature. As mentioned above, modification of an amino acid sequence may be by amino acid substitution, for example an amino acid may be replaced by another that preserves the physicochemical character of the peptide (e.g. A may be replaced by G or vice versa, V by A or L; E by D or vice versa; and Q by N). Generally, the substituting amino acid has similar properties e.g. hydrophobicity, hydrophilicity, electronegativity, bulky side chains etc. to the amino acid being replaced. Isomers of the ‘native’ L-amino acid, e.g. D-amino acids may be incorporated.

Additional variants may include amino and/or carboxyl terminal fusions as well as intrasequence insertions of single or multiple amino acids. Longer peptides may comprise multiple copies of one or more of the peptide sequences. C and N-terminal protecting groups may be included. Insertional amino acid sequence variants are those—in which one or more amino acid residues are introduced at a site in the protein. Deletional variants are characterised by the removal of one or more amino acids from the sequence. “Variants” may include, for example, different allelic variants as they appear in nature, e.g. in other species or due to geographical variation. All such variants, derivatives, or fragments of C-peptide are included, and are subsumed under the term “C-peptide”.

The variants, derivatives and fragments are functionally equivalent in that they have C-peptide activity. More particularly, they exhibit at least 40%, preferably at least 60%, more preferably at least 80% of the activity of proinsulin C-peptide, particularly human C-peptide. Thus they are capable of functioning as proinsulin C-peptide i.e. can substitute for C-peptide itself. Such activity means any activity exhibited by a native C-peptide, whether a physiological response exhibited in an in vivo or in vitro test system, or any biological activity or reaction mediated by a native C-peptide, for example in an enzyme assay or in binding to test tissues or membranes. Thus, it is known that C-peptide increases the intracellular concentration of calcium. An assay for C-peptide activity can thus be by assaying for changes in intracellular calcium concentrations upon addition or administration of the peptide (e.g. fragment or derivative) in question. Such an assay is described in for example Ohtomo et al., (1996), Diabetologia, 39, 199-205; Kunt et al., Diabetes, 47, A30; Shafqat et al., 2002, Cell Mol. Life Sci., 59, 1185-1189. Further, C-peptide has been found to induce phosphorylation of the MAP-kinases ERK 1 and 2 of a mouse embryonic fibroblast cell line (Swiss 3T3), and measurement of such phosphorylation and MAPK activation may be used to assess, or assay for C-peptide activity, as described for example by Kitamura et al., 2001 Biochem J., 355, 123-129. C-peptide also has a well known effect in stimulating Na+K+ATPase activity and this also may form the basis of an assay for C-peptide activity, for example as described in WO 98/13384 or in Ohtomo et al., (1996) or Ohtomo et al., (1998), Diabetologia, 41,287-291. An assay for C-peptide activity based on endothelial nitric oxide synthase (eNOS) activity is also described in Kunt et al., supra, using bovine aortic cells and a reporter cell assay.

Binding to particular cells may also be used to assess or assay for C-peptide activity, for example to cell membranes from human renal tubular cells, skin fibroblasts and saphenous vein endothelial cells using fluorescence correlation spectroscopy, as described for example in Rigler et al., 1999, PNAS USA 96, 13318-13323; Henriksson et al., 2000, Cell Mol. Life Sci 57, 337-342 and Pramanik et al., 2001, BBRC 284, 94-98.

### Therapeutic Methods

A method of treating, preventing or alleviating a symptom of diabetes, e.g., Type I or Type II or disorders associated with diabetes (i.e., diabetic complications) is carried out by administering to a subject in which such treatment or prevention is desired a composition containing insulin and C-peptide in an amount sufficient to treat or prevent the disease in the subject.

Accordingly, in a further aspect the invention also provides a method of combating diabetes and/or a complication thereof, said method comprising administering to a subject in need thereof, a composition of the invention as defined herein.

“Combating” as used herein includes both treatment (in the sense of therapy) and prophylaxis (e.g. of a diabetic complication). In particular, this may include preventing or alleviating a symptom of diabetes. “Diabetes” as defined herein includes any form of diabetes, or any diabetic condition as discussed above, but in particular Type I or Type II diabetes, especially Type I.

Efficaciousness of treatment is determined in association with any known method for diagnosing or treating diabetes. Symptoms of diabetes include fatigue, nausea, frequent urination, excessive thirst, weight loss, blurred vision, frequent infections and slow healing of wounds or sores, blood pressure consistently at or above 140/90, HDL cholesterol less than 35 mg/dL or triglycerides greater than 250 mg/dL, hyperglycemia, hypoglycemia insulin deficiency or resistance. Disorders associated with diabetes include for example, kidney disorders, nerve disorders, vision loss, blindness, heart disease, stroke, or peripheral vascular disease. Alleviation of one or more symptoms indicates that the compound confers a clinical benefit.

The invention also provides methods of increasing serum insulin or C-peptide levels or decreasing blood glucose levels by administering to a subject a composition containing insulin and C-peptide in an amount sufficient to increase serum insulin or C-peptide levels or decrease blood glucose levels. Such a composition may be any composition of the invention as defined herein. Serum glucose levels are decreased or insulin or C-peptide levels are increased in a subject in need thereof. A subject is identified by measuring either blood glucose or insulin levels by methods know in the art. For example by measuring fasting blood glucose levels. A subject is in need of increased serum insulin or decreased blood glucose levels if the subject's insulin or glucose levels are not in normal ranges. Normal adult glucose levels are 60-120 mg/dl. Normal basal insulin levels are 7 μU/mL+3 μU. For example if the subject's serum glucose levels are greater than 120 mg/dl, the subject requires a decrease in serum glucose level. Preferably, after administration the subject's serum glucose is altered to between 60-120 mg/dl. A subject is in need of increased insulin levels, if serum insulin levels are less than 4 μU/mL. Preferably, after administration serum insulin levels are altered such that serum insulin levels are within a normal range, e.g., 5-100 μU/mL.

The subject may be any human or non-human animal but is preferably a mammal. The mammal can be, e.g., a human or a non-human primate, or any pet, or domestic or livestock animal, e.g. a dog, cat, or horse.

Alternatively viewed, this aspect of the invention provides use of insulin and C-peptide for the preparation of a composition for combating diabetes or a complication thereof, or for increasing serum insulin or C-peptide levels or decreasing blood glucose levels, wherein said composition is a composition of the invention as defined herein.

More particularly, this aspect provides use of insulin and C-peptide for the preparation of a composition for combating diabetes or a complication thereof, or for increasing serum insulin or C-peptide levels or decreasing blood glucose levels, wherein said C-peptide is present in said composition at a molar excess of greater than 4-fold with respect to said insulin.

Alternatively, this aspect provides use of insulin and C-peptide for the preparation of a composition for combating diabetes or a complication thereof, or for increasing serum insulin or C-peptide levels or decreasing blood glucose levels, wherein said insulin is an insulin analogue which is not long-acting.

### Therapeutic Administration

The invention includes administering to a subject, e.g. human a composition comprising insulin and C-peptide (referred to herein as a “therapeutic composition”). The composition may be any composition of the invention as defined herein.

An effective daily amount of a therapeutic compound is preferably from about 10-100U insulin and 0.5-3 mg C-peptide. Effective doses vary, as recognized by those skilled in the art, depending on route of administration, excipient usage, and coadministration with other therapeutic treatments including use of other anti-diabetic agents or therapeutic agents for treating, preventing or alleviating a symptom of diabetes. A therapeutic regimen is carried out by identifying a mammal, e.g., a human patient suffering from (or at risk of developing) diabetes using standard methods.

The pharmaceutical compound (active agent) is administered to such an individual using methods known in the art. The compound may be administered orally, rectally, nasally, topically or parenterally, preferably orally, nasally or parenterally e.g., subcutaneously, intraperitoneally, intramuscularly, intravenously and intrapulmonarily. Parenteral administration is preferred, particularly by injection or infusion, e.g. subcutaneous injection, or intravenous infusion. The compound is optionally formulated as a component of a cocktail of therapeutic drugs to treat diabetes and/or a diabetic complication. Examples of formulations suitable for parenteral administration include aqueous solutions of the active agent in an isotonic saline solution, a 5% glucose solution, or another standard pharmaceutically acceptable excipient. Standard solubilizing agents such as PVP or cyclodextrins are also utilized as pharmaceutical excipients for delivery of the therapeutic compounds.

The therapeutic compositions described herein are formulated into compositions for other routes of administration utilizing conventional methods. Such formulations are produced using methods well known in the art.

The therapeutic composition is administered systemically. Additionally, compounds are administered by implanting (either directly into an organ or subcutaneously) a solid or resorbable matrix, which slowly releases the compound into adjacent and surrounding tissues of the subject.

The therapeutic compositions are also administered in conjunction with one or more additional therapeutic compounds such as an anti-diabetic compound. For example, the therapeutic compositions are administered in combination with any of a variety of known therapies for the treatment of disorders associated with diabetes. The additional therapeutic is administered prior to, after or concomitantly with administration of the therapeutic composition.

The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). “Combination therapy” may, but generally is not, intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents.

The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical. “Combination therapy” also can embrace the administration of the therapeutic agents as described above in further combination with other biologically active ingredients and non-drug therapies (e.g., surgery or radiation treatment.) Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

In the methods of the invention, the therapeutic agents are administered in a therapeutically effective amount. A “therapeutically effective amount” of a therapeutic agent refers to the amount needed to achieve a therapeutic objective, such as the treatment of diabetes, or alleviating a symptom associated with diabetes.

In view of the beneficial effect of C-peptide on insulin interaction it is advantageous according to the present invention to formulate the insulin and C-peptide in a single composition for administration. However, this is not absolutely necessary and the benefits of the invention can be achieved by the separate administration of insulin and C-peptide to the same administration site, simultaneously (or substantially simulaneously) or sequentially, or together, eg. by separate injections together or sequentially to the same site, or by providing separate insulin and C-peptide preparations for administration together, eg by infusion or injection together eg. the separate preparations may be mixed just prior to administration. Alternatively, separate injections may be given using two chamber syringes.

Accordingly, in a further aspect the present invention provides a product containing insulin and C-peptide as a combined preparation for simultaneous, separate or sequential use in combatting diabetes or a complication thereof, or for increasing serum insulin or C-peptide levels or decreasing blood glucose levels, wherein said C-peptide is present at a molar excess of greater than 4-fold with respect to said insulin.

In a related aspect the invention provides a product containing insulin and C-peptide as a combined preparation for simultaneous, separate or sequential use in combatting diabetes or a complication thereof, or for increasing serum insulin or C-peptide levels or decreasing blood glucose levels, wherein said insulin is an insulin analogue which is not long-acting.

### EXAMPLES

### Example 1

### General Methods

### Materials

Human insulin (Actrapid) was from Novo Nordisk A/S, Denmark. Proinsulin

C-peptide and scrambled C-peptides (with identical compositions as C-peptide, but with random, highly different amino acid sequences) were synthesized by K J Ross-Petersen, Holte, Denmark. Sensor chip CM5 (research grade), ethanolamine-HCl, N-hydroxysuccinimide (NHS), N-ethyl-N′-[(3-dimethylamino)propyl]carbodiimide hydrochloride (EDC), 2-(2-pyridinyldithio)ethaneamine hydrochloride (PDEA) and surfactant P20 (SP20) were obtained from Biacore (Uppsala Sweden).

### Surface Plasmon Resonance Measurements.

BiacorA Biacore 3000 instrument was employed for the interaction measurements based on surface plasmon resonance (SPR). Interaction analyses were performed at 25° C. in 10 mM Na-citrate buffers pH 3, 4, 5, 10 mM bis-Tris, pH 6, and 10 mM Tris/HC1, pH 7, all buffers containing 100 mM NaCl and 0.005% 5P20. Flow rates for the binding analyses were kept at 20 μl/min. Insulin was desalted and buffer was exchanged on NAP 10 columns from Amersham Biosciences.

Working insulin solution was prepared after removing the excess of zinc sulfate, meta-cresol and other salts from the original solution by passing through a NAP 10 column in the desired buffer for experiments. The desalted insulin was diluted to 1 mM, aliquoted and stored at −20° C. The resulting solution was found to be a mixture of monomers, dimers and hexamers as observed by electrospray ionization with a Q-TOF mass spectrometer.

Insulin was immobilized on a carboxymethyl-modified dextran surface via both amine and carboxyl coupling, using the surface thiol method, in two different lanes of Biacore CM5 chips. The immobilization was carried out according to the procedure described in the kits. In brief, the sensor chip surface was first washed with 4 M LiCl/HBS for 1 min and then with 0.2% SP20/HBS for 1 mM. The surface was activated according to standard procedure with an injection of 0.05 M NHS/0.2 M EDC for 7 min. Desalted insulin solution was diluted in acetate buffer, pH 4.5, to 20 μg/ml and injected at 50/min for different time intervals. For surface thiol coupling, the surface was first activated with 0.05 M NHS/0.2 M EDC for 2 min followed by a 3 min injection of 40 mM cystamine dihydrochloride and then 0.1 M DTE for 3 min. At the same time, 2-(2-pyridinyldithio)ethaneamine hydrochloride (PDEA) insulin was prepared by dissolving 0.5 mg insulin in 0.5 ml 0.1 M MES, adding to it 0.25 ml of (15 mg/ml) PDEA in 0.1 M MES, pH 5, and 25 μl 0.4M EDC. The mixture was incubated for 10 mM at room temperature and excess PDEA was removed on a NAP-10 column. The purified PDEA ligand was injected for 7 min, soon after DTE, and finally the surface was washed with 20 mM PDEA in 0.1 M sodium acetate, 1.0 M NaCl. For quick regeneration of the surface, 2 M guanidine-HC1, pH 6.4, was employed. For controls, lanes activated and deactivated according to the standard procedures were used. For subtraction of noise from the data, running buffer only was injected to all surfaces.

For the C-terminal attachment, surface-thiol coupling to 4000-5000 RU was used, and for N-terminal attachment, amine coupling to 800-1000 RU as achieved on different CM5 chips. However for kinetic analysis immobilization was carried out in a controlled condition and around 1000 RU for C-terminal and 400 RU for N-terminal coupling were immobilized. A surface activated with EDC/NHS and deactivated with ethanolamine was used as a control surface for N-terminally immobilized insulin and a surface treated with EDC/NHS, cysteine/DTE and finally deactivated with PDEA-NaCl was used as a control for C-terminally immobilized insulin.

Immobilization of insulin on CM5 chip was strictly monomeric when immobilized by N-terminal attachment using amine coupling, since some dimers and higher complexes were removed when the surface was washed with 0.1 M ethanolamine after immobilization. About a 40% drop in immobilization level was then observed, after which the surface binding was found to be stable. Immobilization via C-terminal attachment, using surface thiol coupling, showed somewhat higher immobilization levels which were not changed by injection of PDEA-NaCl or 2 M guanidine-HCl, pH 6.4.

Biacore data were analyzed with Bia-evaluation software 4.1. All calculations based on numerical approaches, including simulations and global curve fitting for estimates of active concentrations, were performed with IGOR Pro (version 4.01.A, WaveMetrics, Inc.).

### Mass Spectrometry

For mass spectrometric analysis, preparation of concentrated insulin stock solution was performed essentially as described [Nettleton et al, 2000, Biophys J. 79:1053]. Briefly, insulin (NovoNordisk A/S, Denmark) was concentrated to 100 μM using ultrafiltration spin-filters (NanoSep, PallGellman, MWCO: 3000 Da). This stock solution was then buffer-exchanged to water, pH 5 (HCl), by extended dialysis. For all experiments, insulin and C-peptide stock solutions were diluted in water-HC1 (pH 5) to reach the indicated concentrations. C-peptide was prepared as a 746 μM stock solution in water and all control peptides were prepared similarly.

Mass spectrometry data were recorded with a QTOF Ultima API instrument (Waters, Milford, Mass.) operated in the positive ion mode. Sample solutions were analyzed by nanoelectrospray from conductive borosilicate glass capillaries (Proxeon Biosystems A/S, Odense, Denmark). Instrument settings were not modified for the observation of non-covalent complexes, other than that the “cone” and the “RF-lens” settings were set to 90 and 50 V, respectively.

### Example 2

### Insulin-Insulin Interactions

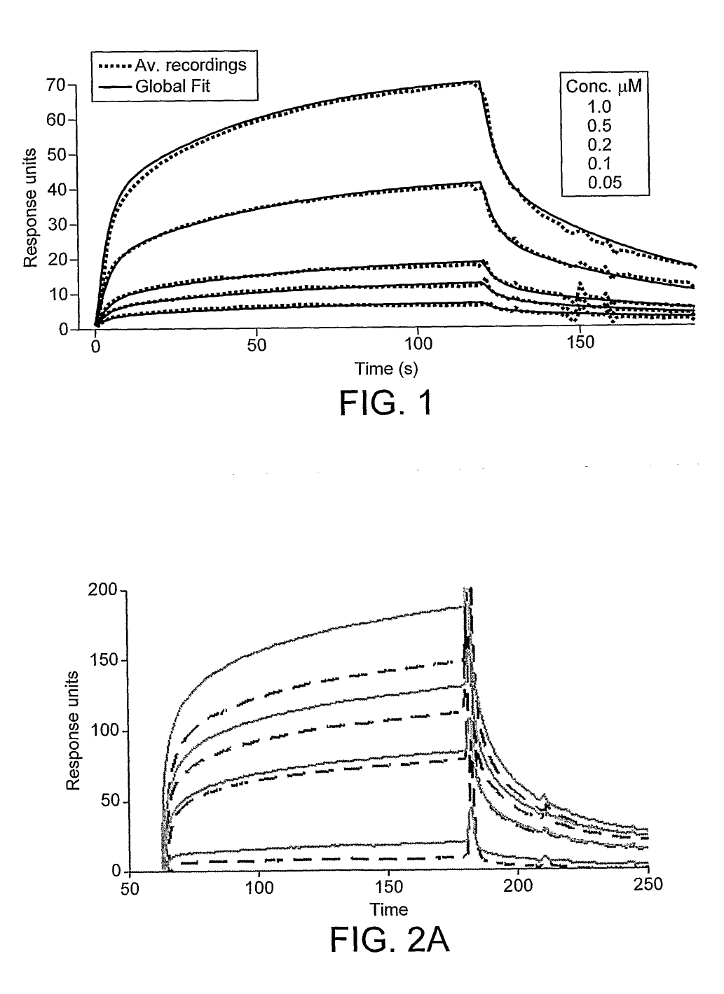

Interactions were studied by surface plasmon resonance using a Biacore 3000 instrument with CM5 chips having both C- and N-terminally immobilized insulin in two different lanes. Results clearly showed that free insulin interacts with insulin molecules immobilized on a dextran surface of CM5 chip independent to the direction of immobilization. For kinetic analysis, insulin in different concentrations ranging from 0.25-10 μM were applied, each concentration was run in triplicate and in random order. Background noise was removed after subtracting the average values of running buffer injected three times. Non-specific surface interaction of insulin was removed by subtraction of the blank lanes in each run. After all subtractions and averaging of the curves in the Bia evaluation program, the data obtained were globally fitted by using a least-squares curve fitting procedures with IGORE pro. The models used to interpret the insulin-insulin interaction includes: 1) one to one binding, 2) dynamic dimer binding and 3) analyte with two binding site competing for one binding site on the chip. The data used for fitting was from C-terminally immobilized insulin. For the first two models the data did not fit well but it agreed fully with the third model. This means that the insulin in solution has two possibilities to interact with immobilized insulin, which has only one of its two sites free. From the curves it is also possible to calculate two dissociation constants; K_{D1}=2.71 μM and K_{D2}=4.65 μM, representing slow and rapid off rates, respectively very quick off. (FIG. 1)

These results show that the insulin/insulin interactions can be studied by SPR and that each monomer has two binding sites, of which one is blocked by the N- and C-terminal attachments respectively, on the chip-bound monomer.

### Example 3

### Effect of C-Peptide on Insulin-Insulin Interactions

Proinsulin C-peptide showed no interaction with insulin when passed over insulin immobilized on the surface of CM5 chip. Similar results where obtained when insulin was injected on C-peptide immobilized on the surface of a streptavidin (SA) chip. Both peptides were injected in concentration ranging from pM-μM at different pH from 3-7. These results show that C-peptide and insulin monomers appear to have no strong binding site for each other. An alternative interpretation, that they might have a strong monomer/monomer interaction site (but blocked by the attachment of the peptides on the chip) appears less likely for two reasons: first, a site is created by addition of a second insulin monomer, and this monomer is identical to the chip-bound insulin monomer but still gives a site; second, ESI-MS, shows little heterodimers, although insulin hexamers are observed. Hence, C-peptide is concluded to lack a strong binding site for an insulin monomer. This conclusion is compatible with the fact that C-peptide and insulin are joined in proinsulin by covalent bonds rather than binding attachments, and with little ordered structure in the C-peptide part. We also tested binding of C-peptide in solution to chip-bound C-peptide and found no evidence for such binding either. This appears relevant since C-peptide is very negative (−6 charges) with much repulsive forces.

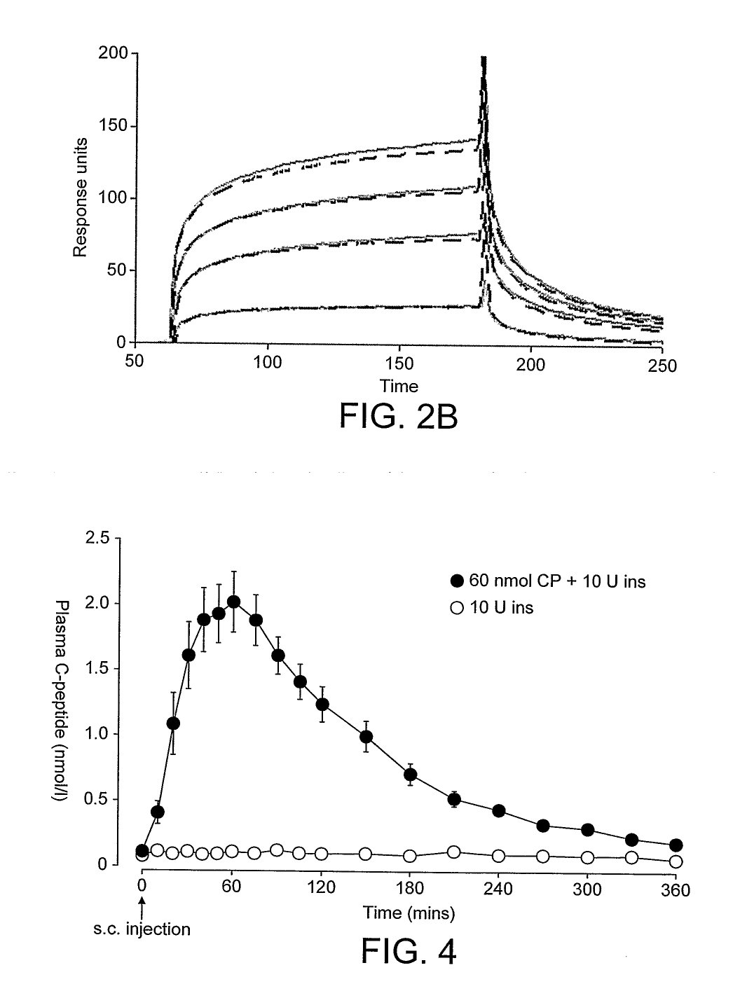

However, C-peptide, mixed with insulin in solution, influences the insulin-insulin monomer interactions measured above. When C-peptide was mixed with insulin in 1:1 and 1:5 ratio, the mixture showed higher interaction with immobilized insulin as compared to insulin alone. This elevated interaction can be seen at different pH from 3-7. The phenomenon of more pronounced interaction was clearer when concentrations higher than 0.5 μM were used then below 0.5 μM. FIG. 2A shows insulin-insulin interactions at different concentrations both in absence and presence of 5×C-peptide. All concentrations were run in triplicates and in random order. A scrambled C-peptide in similar conditions and concentrations failed to show significant increase in insulin-insulin interaction (FIG. 2B).

### Example 4

### Effect of pH on Insulin-Insulin Interaction

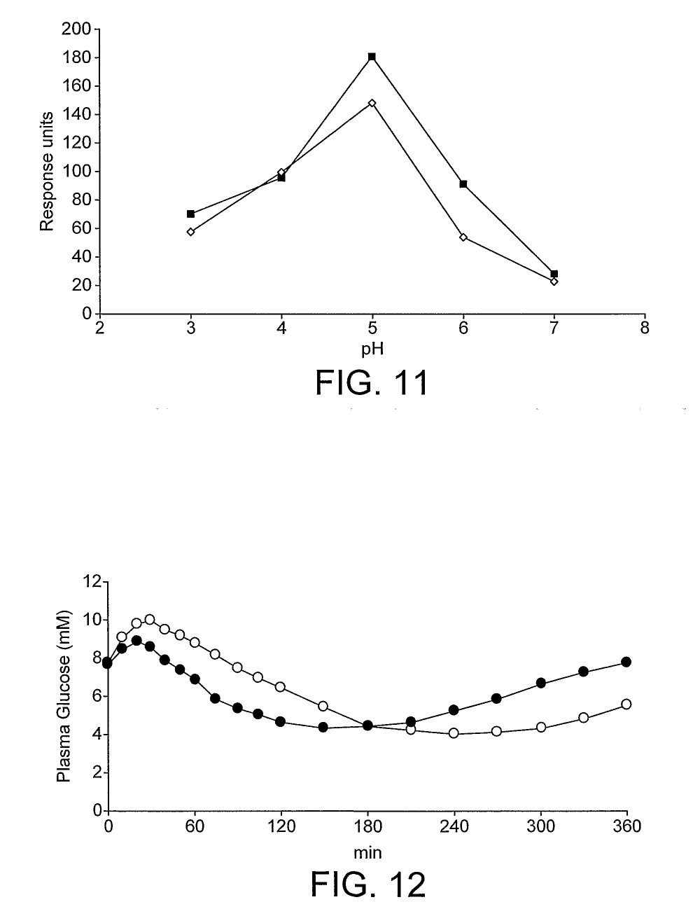

The influence of pH on insulin-insulin interaction has been studied in the pH range 3-7. The highest binding interaction was seen at pH 5 with lower values being observed at pH 3-4 and 6-7 (FIG. 11). Enhanced insulin interaction is seen in the presence of C-peptide at pH 5-6, while little effect of C-peptide was observed at pH 4 or 7.

### Example 5

### Effect of C-Peptide Variants on Insulin-Insulin Interaction

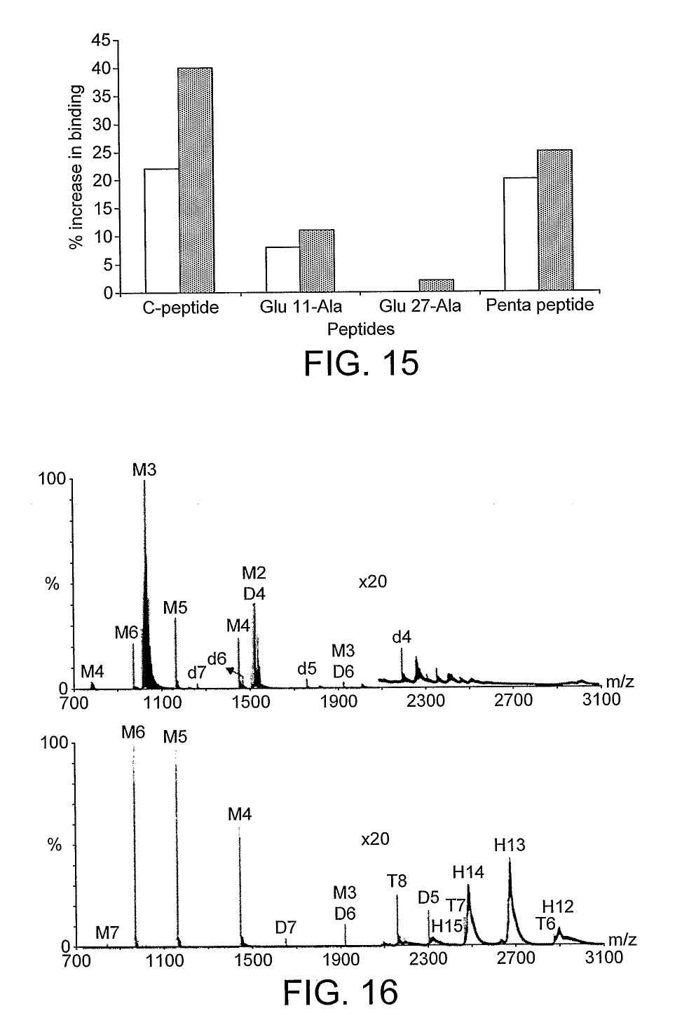

C-peptide variants having Glu11-Ala, Glu27-Ala and Glu3, 11 and 27 all exchanged for Ala when injected along with insulin in 1:5 molar ratios failed to show any significant increase in insulin-insulin interaction, attesting to the specificity of C-peptides effect. These peptides showed no interaction with insulin. In FIG. 15, it can also be seen that the C-terminal penta peptide was found to be as equally effective as the native C-peptide in enhancing insulin-insulin binding at a 1:1 molar ratio.

### Example 6

### Effect of Zinc and EDTA on Insulin-Insulin Interaction

Addition of zinc sulfate in molar ratios higher then insulin resulted in a diminished insulin interaction while presence of EDTA promoted insulin-insulin interaction at concentrations up to 10 μM.

### Example 7

### Mass Spectrometric Results

The binding between insulin and C-peptide in the gas-phase was studied by non-denaturing electrospray mass spectrometry using a method analogous to that of Nettleton et al [Nettleton et al, 2000, Biophys J. 79:1053]. The mass spectrometric data obtained by nanoESI clearly shows the interaction between insulin and C-peptide at 3 μM and above when both peptides were mixed in 1:1 ratio. Interaction at the 0.3 μM level was not possible to detect by this method. Already at 3 μM level spectra showed a series of multiple charged states of the complex among them +7, +6, +5, and +4 were dominant and correspond to an average molecular mass of 8827 Da which is very close to the calculated mass of the complex. A scrambled C-peptide and another differently scrambled C-peptide (S2) under similar conditions showed only a very weak interaction at the 3 μM level. In 1:5 molar ratio (30 μM insulin and 150 μM peptides) both scrambled and S2 showed an interaction with insulin, but this interaction was weaker compared to that of C-peptide.

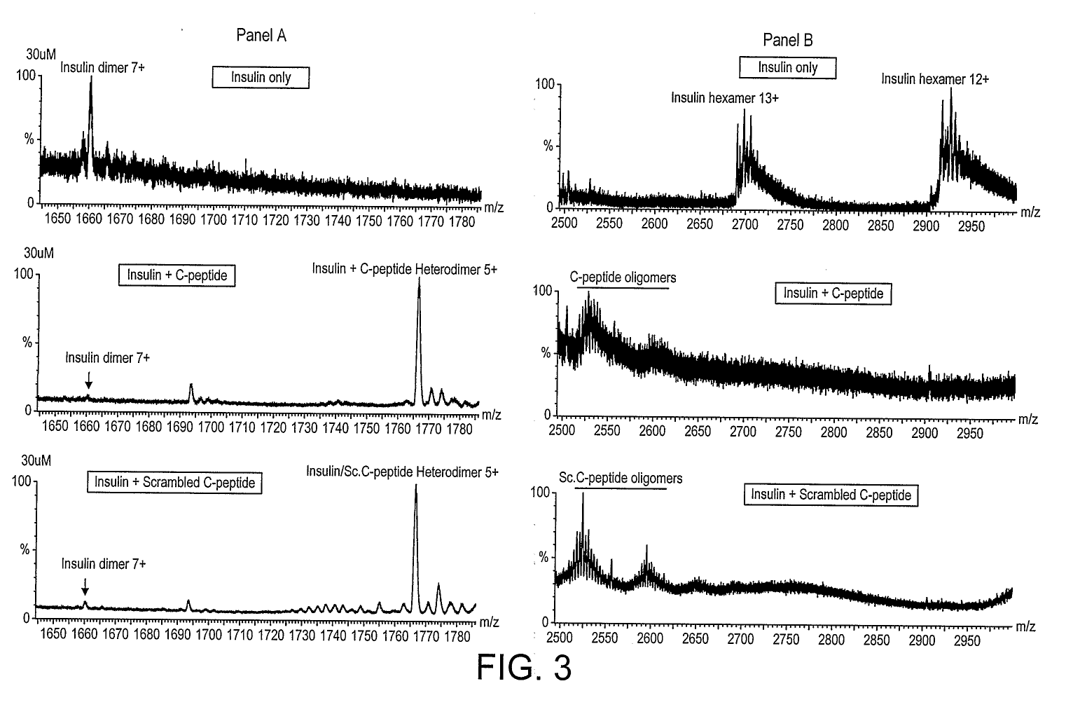

Under the conditions used, the hexameric state of insulin was also found to be markedly influenced. Thus in the presence of C-peptide, no hexameric insulin was detectable (FIG. 3 and FIG. 16).

C-peptide and scrambled C-peptide both showed an aggregation tendency in the gas-phase and already at 3 μM, a dimer of C-peptide was detected and at higher concentrations such as 15 μM, 30 and 150 μM other species such as dimers, trimers, tetramers and hexamers were found.

In conclusion, therefore, the presence of hexamer signals at different charge states in the ESI mass spectra decreased from their level in insulin solutions to an absence in insulin/C-peptide mixtures (FIG. 3B). Hence, ESI-MS analysis suggests that C-peptide depolymerizes the hexameric aggregates of insulin.

Combined, the SPR and ESI MS data show that C-peptide influences insulin/insulin interactions, affecting in particular oligomeric states, and at least in ESI-MS decreasing hexameric signals. As obvious from the presence of several oligomers, there should be many separate interaction constants. Two were estimated by curve fitting to binding models in the SPR experiments, both in the μmolar range. At that range, interactions are probably not of interest in serum or tissues, but other interactions with further binding can exist. The μmolar constants measured are of interest in relation to insulin secretion in the pancreas of healthy individuals and at the injection sites of diabetic subjects. Thus, it is considered that a physiological effect of C-peptide may be that it contributes to the formation of insulin monomers following exocytosis of the secretory granules' content of hexameric insulin into the extracellular space. Similarly, it is further proposed that C-peptide may affect insulin oligomeric states and desaggregations at subcutaneous sites of insulin injection in diabetic patients.

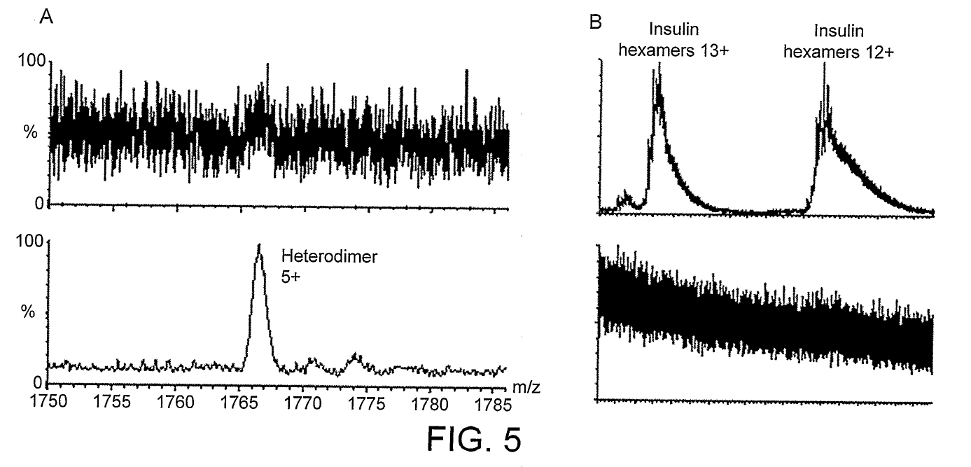

Reference is also made to FIG. 5 which shows the nano-ES mass spectra for C-peptide, scrambled C-peptide and insulin. The amplification in FIG. 5, Section B, bottom panel is enhanced to demonstrate the absence of hexamers.

### Example 8

### Absorption Kinetics and Metabolic Effects of Combined C-Peptide and Insulin Injection

The metabolic effects of combined C-peptide and insulin injection were studied in nine Type 1 diabetic patients. Their individual characteristics are given in Table 1. Apart from insulin they had no other medication. All patients received injections of soluble insulin before meals and intermediate-acting insulin at bedtime. Six patients had simplex retinopathy and two of these (nos. 4 and 8) also had microalbuminuaria. All subjects were informed of the purpose, nature and possible risks before giving their consent to participate in the study, which was approved by the local Ethics Committee.

### Procedure

The experiments were carried out on two days separated by at least one week. The patients were admitted to the hospital at 8 p.m. in the evening before the study day. They received their last dose of insulin at 4-6 p.m. In order to clear the subcutaneous depots of insulin no intermediate-acting insulin was given at bedtime. Instead, an intravenous infusion of insulin was started at the time of admission. Blood glucose was measured every 1-2 hour, and the rate of insulin infusion was adjusted to achieve a blood glucose concentration of 4-7 mmol/l the following morning. On the study day the investigation started at 7.30 a.m. after an overnight fast. A short teflon catheter was inserted into an antecubital vein for blood sampling. The insulin infusion was stopped and after 20 min blood was sampled for the analysis of plasma glucose, free insulin, C-peptide free fatty acids (FFA) and glucagon. Thereafter, injections of either biosynthetic human C-peptide (60 nmol), a gift from Eli Lilly (Indianapolis, USA), plus an equimolar amount of soluble insulin (CI-day; Humulin; 10U; Eli Lilly, Indianapolis, USA) or soluble insulin and C-peptide diluent (I-day), were injected into the abdominal wall in a randomized double blind fashion. The injections were given with an automatic injection device (which allowed standardization of speed and depth) approximately 100 mm lateral to the umbilicus into the middle of the subcutaneous tissue, the depth of which was predetermined with ultrasound.

After the injections plasma glucose was allowed to decrease to 3.5 mmol/l, whereafter it was maintained at 3.5-5 mmol/l using a variable glucose infusion (DeFronzo R, Tobin J, Andres R, American Journal of Physiology 237:E214-E223, 1979) The patients were followed for 6 hours. Blood samples for the determination of plasma glucose, free insulin, C-peptide, FFA and glucagon were taken at timed intervals.

### Biochemical Analyses

Plasma glucose was measured with a glucose oxidase method on a Beckman Glucose Analyzer 2 (Beckman, Fullerton, Calif., USA). Plasma free insulin was determined by radioimmunoassay after immediate polyethylene glycol precipitation (Arnquist H, Olsson P, von Schenk H, Clinical Chemistry 33:93-96, 1987). C-peptide (Heding L, Diabetologia 11:541-548, 1975) and glucagon were also analyzed by radioimmunoassay using a commercially available kit (MILAB, Malmö, Sweden). Plasma FFA (Hoogwerf B, Bantle J, Gaenslen H, Greenberg B, Senske B, Francis R, Goetz F, Metabolism 35:122-125, 1986) was determined by a fluorometric method.

### Statistics and Data Analysis

Standard statistical methods including Wilcoxon's nonparametric test, Student's paired t-test and linear regression analyses were performed when applicable. Results are presented as mean values±SEM. P values <0.05 were considered to be significant. The areas under the plasma insulin, and free fatty acid concentration curves were computed by trapezoidal integration.

### Plasma C-Peptide

The average basal plasma C-peptide concentration was 0.11±0.03 nmol/l on both the CI-day and the I-day. Already at 10 min after the CI-injection plasma C-peptide measured 0.44±0.10 nmol/l, which was significantly higher than on the I-day (FIG. 4, p<0.01 between days). Peak plasma levels of 2.09±0.25 nmol/l were seen after 57±3 min on the CI-day, whereas the basal plasma C-peptide levels were unaltered on the I-day, as could be expected. Physiological plasma concentrations of C-peptide (>0.3 nmol/l) were maintained up to 300 min after the CI-injection, and at 360 min C-peptide levels were still significantly higher than on the I-day measuring, 0.20±0.02 vs. 0.10±0.02 nmol/l (p<0.01; FIG. 4).

### Plasma Immunoreactive Insulin

Basal plasma immunoreactive insulin (IRI) concentrations measured 4±1 mU/l and 5±1 mU/l on the CI- and I-day, respectively. Peak plasma IRI of 27±4 mU/1 and 28±3 mU/1 were found after 88±14 min and 88±11 min on the CI and I-day, respectively. The areas under the plasma IRI curves (0-360 min) were similar during the two study days, measuring 5570±419 and 5847±430 mU/l×min on the CI and I-day, respectively.

### Plasma Glucose

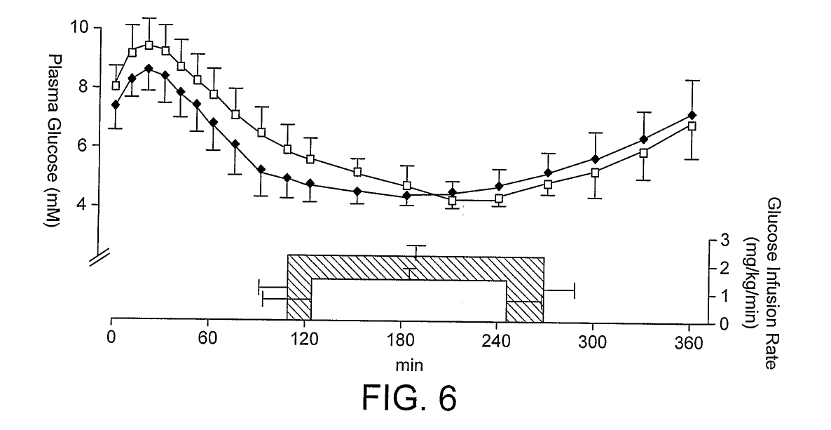

Prior to cessation of the overnight insulin infusion plasma glucose measured 6.7±0.5 and 6.9±0.5 mmol/l on the CI and I-day, respectively. Twenty min later, when the subcutaneous injections of CI or I were given, the corresponding values were 7.3±0.6 and 8.1±0.6 mmol/l (FIG. 6).

Subsequently, the plasma glucose concentration declined and in order to avoid hypoglycemia 8 of the 9 patients on the CI-day and 7 of 9 patients on the I-day required intravenous infusion of glucose to avoid hypoglycemia. Plasma glucose measured 3.6±0.07 and 3.5±0.05 mmol/l, respectively, at the beginning of the glucose infusions. The glucose infusion tended to be of longer duration on the CI-day compared to the I-day, measuring 158±24 min vs. 124±28 min, respectively. The required glucose infusion rate was higher (p<0.05) and the total amount of infused glucose needed to prevent hypoglycemia was 66% greater on the CI-day compared to the I-day (p<0.02), indicating an increased whole body glucose utilisation on the CI-day FIG. 6.

### Plasma FFA

Basal FFA levels did not differ on the two study days; they were 0.52±0.11 vs. 0.73±0.12 mmol/l on the CI and I-days, respectively. An increase to 0.74±0.09 and 0.80±0.11 mmol/l was seen at 30 min after the cessation of the i.v. insulin infusion (10 min after the CI and I injections). Subsequently, the FFA levels decreased, reflecting inhibition of lipolysis in adipose tissue. After correction for the plasma free insulin area the AUC for the plasma FFA concentration during 0-180 min was significantly smaller on the CI-than on the I-day measuring 48.811.2 vs. 55.6±5.1 mmol/l×min (p<0.01; FIG. 7, reflecting a more marked anti-lipolytic effect on the CI-day. For the study period 0-360 min, the corresponding values were 123±20 and 129±17 mmol/l×min, respectively.

### Plasma Glucagon

The basal plasma glucagon concentrations were similar on the two study days (50±2.5 pg/ml and 52±2.7 pg/ml on the CI-day and I-day, respectively). Following the injection on the CI-day glucagon levels tended to be lower and the area under the concentration curve was smaller (FIG. 8 than on the I-day.

### Example 9

### Effect of C-Peptide on Insulin-Insulin Interactions (SPR Analysis)

As reported above in Example 3, a 1-5 fold C-peptide excess over insulin analyte monomer increases the observable SPR binding signal (FIG. 2A), and this effect is reproducible over a considerable concentration interval. It is also fairly specific for C-peptide, not shown to an appreciable extent by scrambled C-peptide (FIG. 2B). Whether the added binding is due to additional insulin monomer binding to the insulin oligomer (FIG. 1, and the gray of FIG. 2A), or to novel C-peptide binding once insulin oligomer(s) have been formed, is unknown from these experiments. However, the binding increase by the presence of C-peptide with the insulin appears smaller than the binding increase by just addition of the same amount of extra insulin. Also, the new curve obtained in the presence of C-peptide does not fit into the standard model fittings tested above. Presumably therefore, the extra interaction observed in the presence of C-peptide and insulin, may involve oligomer states of insulin, and likely also some binding interactions of C-peptide with any such state. In further testing, we found that time and temperature had some, but limited effects, on the binding curves. Thus, higher temperature (37° instead of) 25° increased the insulin/insulin binding, while time slowly decreased it and C-peptide then appeared to slightly reduce the rate of decrease. In conclusion, C-peptide appears to influence insulin oligomer binding capacity and to some extent oligomer stability.

### Example 10

### Further Tests with C-Peptide Variants

We further tested C-peptide analogues. The C-terminal pentapeptide, previously shown to replace C-peptide effects in several assays, also stimulated SPR-measurable binding when in mixture with insulin in the flow-through solution, while a Glu27Ala C-peptide analogue, inactive in the assays, did also not stimulate the SPR signal in the presence of insulin. Two other analogues with Glu to Ala replacements (at positions 3 and 11), previously found to be more active in one assay than the position 27 replacement, also gave more SPR signal increase with insulin than did the position 27 analogue. In conclusion, it appears that much of the insulin oligomer influence as now measured rests with the presence of Glu27, whether in the C-terminal pentapeptide or in the whole of C-peptide. Hence, it may be concluded that the importance of Glu27 appears to be correlated with the C-peptide effect on SPR measurable binding to insulin oligomers.

### Example 11

### Absorption Kinetics and Effects of Combined C-Peptide and Insulin Injection as Compared with Separate Injections

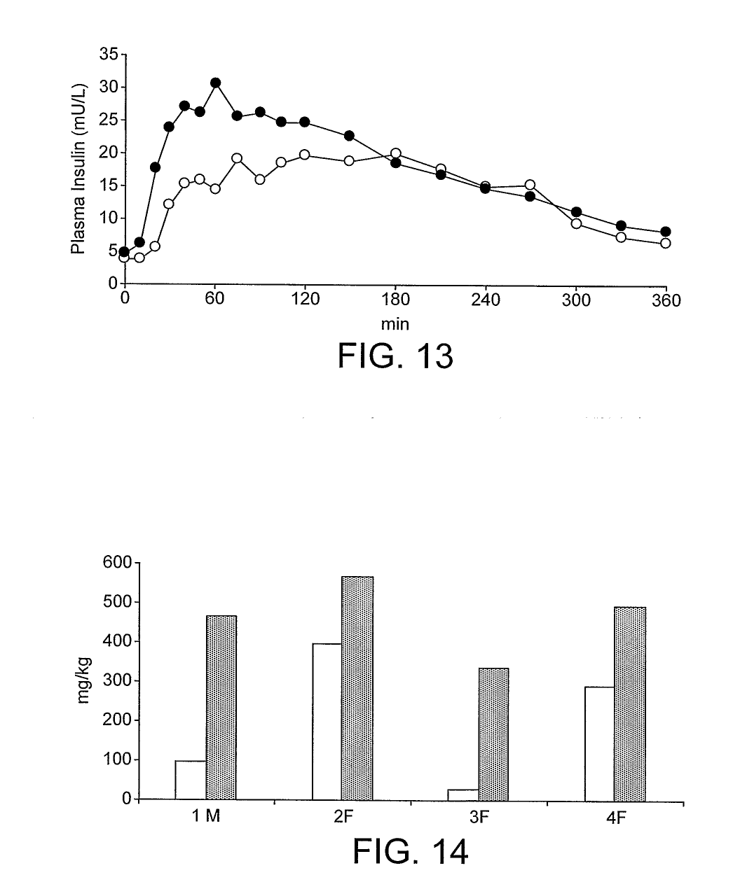

In four patients we did clamp studies following injection of insulin and C-peptide in equimolar amounts either in a combined subcutaneous injection at one site or as separate but simultaneous injections at two different sites to the right and left of the umbilicus, respectively, about 20-25 cm apart. The clamp was done as in the other patients as described in Example 8. The ensuing glucose concentration changes are shown in FIG. 12. The fall in glucose concentrations from 0-60 min (P<0.02) and from 0-120 min (P<0.03) was significantly more marked after the combined injection compared to after separate injections. The appearance of insulin in plasma was more rapid and the total AUC was greater after combo injection compared to after separate injections (AUC 0-360 min P<0.05), (AUC 0-60 min, P<0.07), see FIG. 13. Finally, the total amount of glucose that needed to be infused in order to prevent hypoglycemia is shown in FIG. 14 for each of the four subjects. The duration of glucose infusion was longer after the combined injection 186+/−24 min vs 117+/−36 min P<0.02 than separate injections. Likewise, the average total amount of glucose that had to be infused was larger after combined injection (467+/−48 mg/kg vs 204+/−85 mg/kg, P<0.01).

This supports the hypothesis that C-peptide, when in direct contact with the hexameric insulin that comes out the vial, facilitates its desaggregation into dimers and eventually monomers, the biologically active form.

TABLE 1
Individual clinical data for the patients

| Patient | Sex | Age | BMI | Diabetes duration | Insulin dose | HbAlc* | C-peptide |
|---|---|---|---|---|---|---|---|
| number | (female, male) | (years) | (kg/m²) | (years) | (U/kg/24 h) | (%) | (nmol/l) |
| 1 | m | 46 | 26.9 | 31 | 0.95 | 5.2 | 0.15 |
| 2 | m | 28 | 21.9 | 13 | 0.78 | 7.7 | 0.18 |
| 3 | m | 32 | 24.3 | 12 | 0.89 | | <0.10 |
| 4 | f | 29 | 26.0 | 15 | 0.40 | 9.9 | 0.19 |
| 5 | f | 23 | 26.7 | 12 | 0.76 | 6.1 | 0.18 |
| 6 | f | 29 | 20.6 | 11 | 0.70 | 5.8 | <0.10 |
| 7 | f | 35 | 22.5 | 22 | 0.75 | 5.1 | 0.19 |
| 8 | f | 34 | 21.2 | 20 | 0.80 | 6.5 | <0.10 |
| 9 | m | 31 | 26.3 | 17 | 0.63 | 8.3 | <0.10 |

Means ± SEM are given
*normal value <4%

Other embodiments are within the following claims.

1. A composition comprising insulin or analogue thereof, a C-peptide or analogue thereof in a ratio on a molar basis of about 1:1 to 1:5 and a carrier wherein said composition is at a pH of 5.0-7.0.

2. The composition of claim 1, wherein said insulin is human insulin.

3. The composition of claim 1, wherein said insulin is recombinant insulin.

4. The composition of claim 1, wherein said insulin is chemically synthesized.

5. The composition of claim 1, wherein the pH is 5.0-6.7

6. The composition of claim 1, wherein the pH is 5.0-6.0.

7. The composition of claim 1, wherein the pH is 5.0.

8. The composition of claim 1, wherein the ratio of insulin to C-peptide is 1:1.

9. The composition of claim 1, wherein the ratio of insulin to C-peptide is 1:5.

10. The composition of claim 1, wherein said composition does not contain zinc.

11. The composition of claim 1, wherein said composition when administered to a subject increases the insulin sensitivity index of said subject at least 10% greater than in a subject administered insulin alone.

12. A method of treating or alleviating a symptom of diabetes comprising identifying a subject suffering from diabetes and administering to said subject the composition of claim 1.

13. The method of claim 12, wherein said subject is human.

14. The method of claim 12, wherein said diabetes is Type I diabetes or Type II diabetes.

15. The method of claim 12 wherein the insulin sensitivity index in said subject is at least 10% greater than in a subject administered insulin alone.

16. A method preventing insulin aggregation in vivo comprising administering to a subject in need thereof the composition of claim 1.

17. The method of claim 16, wherein said subject is human.

18. A composition formulated to prevent insulin aggregation, comprising insulin or analogue thereof, and a C-peptide or analogue thereof.

19. The composition of claim 18, wherein said insulin and said C-peptide is at a ratio on a molar basis of about 1:1 to 1:5

20. The composition of claim 18, wherein said composition is at a pH of 5.0-7.0.

21. (canceled)

22. (canceled)

23. (canceled)

24. (canceled)

25. A composition comprising insulin and C-peptide together with at least one pharmaceutically acceptable excipient or carrier wherein said C-peptide is present at a molar excess of greater than 4-fold with respect to said insulin.

26. The composition of claim 25, wherein the molar ratio of insulin:C-peptide is 1:5 or more.

27. The composition of claim 25, wherein the molar ratio of insulin:C-peptide is in the range 1:4.2 to 1:10.

28. A composition comprising an insulin analogue and C-peptide, together with at least one pharmaceutically acceptable carrier or excipient, wherein said insulin analogue is not a long-acting analogue.

29. The composition of claim 28 wherein said insulin analogue is Aspart, Lispro or Insulin Glulisine.

30. The composition of any of claims 25 to 29 wherein the pH of the composition is from pH 5.0 to 8.0

31. The composition of claim 30, wherein the pH is from pH 5.0 to 6.5 or pH 5.0 to 7.5.

32. (canceled)

33. (canceled)

34. A product containing insulin and C-peptide as a combined preparation for simultaneous, separate or sequential use in combatting diabetes or a complication thereof, or for increasing serum insulin or C-peptide levels or decreasing blood glucose levels, wherein said C-peptide is present at a molar excess of greater than 4-fold with respect to said insulin.

35. A product containing insulin and C-peptide as a combined preparation for simultaneous, separate or sequential use in combatting diabetes or a complication thereof, or for increasing serum insulin or C-peptide levels or decreasing blood glucose levels, wherein said insulin is an insulin analogue which is not long-acting.


SEQUENCE LISTING (33 sequences)

SEQ ID NO: 1 - protein, 31 aa, Homo sapiens
EAEDLQVGQVELGGGPGAGSLQPLALEGSLQ

SEQ ID NO: 2 - protein, 5 aa, Artificial Sequence
  Pentapeptide of C-peptide
EGSLQ

SEQ ID NO: 3 - protein, 31 aa, Artificial Sequence
  Generic sequence with conserved Glu residues
  VARIANT 1: Xaa is E or G
  VARIANT 2, 4, 5, 7-10, 13-24, 28-30: Xaa any amino acid
XXEXXQXXXXELXXXXXXXXXXXXALEXXXQ

SEQ ID NO: 4 - protein, 12 aa, Artificial Sequence
  N-terminal fragment
EAEDLQVGQVEL

SEQ ID NO: 5 - protein, 31 aa, Pan troglodytes; identical to SEQ ID NO: 1

SEQ ID NO: 6 - protein, 29 aa, Aotus trivirgatus
EAEDLQVGQVELGGGSITGSLPPLEGPMQ

SEQ ID NO: 7 - protein, 32 aa, Macaca fascicularis
EAEDPQVGQVELCSGGPGAGSLQPLALEGSLQ

SEQ ID NO: 8 - protein, 31 aa, Cercopithecus aethiops
EAEDPQVGQVELGGGPGAGSLQPLALEGSLQ

SEQ ID NO: 9 - protein, 29 aa, Sus sp.
EAENPQAGAVELGGGLGGLQALALEGPPQ

SEQ ID NO: 10 - protein, 26 aa, Artificial Sequence
  Boven C-peptide
EVEGPQVGALELAGGPGAGGLEGPPQ

SEQ ID NO: 11 - protein, 31 aa, Equus sp.
EAEDPQVGEVELGGGPGLGGLQPLALAGPQQ

SEQ ID NO: 12 - protein, 26 aa, Ovis sp.
EVEGPQVGALELAGGPGAGGLEGPPQ

SEQ ID NO: 13 - protein, 31 aa, Canis sp.
EVEDLQVRDVELAGAPGEGGLQPLALEGALQ

SEQ ID NO: 14 - protein, 30 aa, Oryctolagus cuniculus
EVELQVGQAELGGGPGAGGLQPSALELALQ

SEQ ID NO: 15 - protein, 29 aa, Rattus sp.
EVEDPQYPQLEGGPEAGDLQTLALEVARQ

SEQ ID NO: 16 - protein, 31 aa, Rattus sp.
EVEDPQVAQLELGGGPGAGDLQTLALEVARQ

SEQ ID NO: 17 - protein, 31 aa, Unknown
  rodent C-peptide
EVEDPQVGQVELGAGPGAGSEQTLALEVARQ

SEQ ID NO: 18 - protein, 29 aa, Mus sp.
EVEDPQVEQLELGGSPGDLQTLALEVARQ

SEQ ID NO: 19 - protein, 31 aa, Mus sp.
EVEDPQVAQLELGGGPGAGDLQTLALEVAQQ

SEQ ID NO: 20 - protein, 31 aa, Cavia porcellus
ELEDPQYEQTELGMGLGAGGLQPLALEMALQ

SEQ ID NO: 21 - protein, 31 aa, Unknown
  crilo C-peptide
GYEDPQVAQLELGGGPGADDLQTLALEVAQQ

SEQ ID NO: 22 - protein, 31 aa, Unknown
  psaob C-peptide
GYDDPQMPQLELGGSPGAGDLRALALEVARQ

SEQ ID NO: 23 - protein, 29 aa, Unknown
  Ocide C-peptide
ELEDLQVEQAELGLEAGGLQPSALEMILQ

SEQ ID NO: 24 - protein, 18 aa, Mus spretus
GGPGAGDLQTLALEVAQQ

SEQ ID NO: 25 - protein, 16 aa, Mus spretus
GSPGDLQTLALEVARQ

SEQ ID NO: 26 - protein, 28 aa, Anas sp.
  Xaa any amino acid
DVEQPLVNGPLKGEVGELPPQHEEYQXX

SEQ ID NO: 27 - protein, 28 aa, Gallus sp.
DVEQPLYSSPLKGEAGYLPPQQEEYEKV

SEQ ID NO: 28 - protein, 30 aa, Homo sapiens
FVNQHLCGSHLVEALYLVCGERGFFYTPKT

SEQ ID NO: 29 - protein, 21 aa, Homo sapiens
GIVEQCCTSICSLYQLENYCN

SEQ ID NO: 30 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 31 - protein, 4 aa, Artificial Sequence
  C-peptide fragment
ESLQ

SEQ ID NO: 32 - protein, 21 aa, Artificial Sequence
  insulin analog A chain
  VARIANT 21: Xaa is A, N, D, Q, E, G, T or S
GIVEQCCTSICSLYQLENYCX

SEQ ID NO: 33 - protein, 32 aa, Artificial Sequence
  insulin analog B chain
  VARIANT 1: Xaa is F, D or is absent
  VARIANT 2: Xaa is V or is absent when Xaa ar position 1 is absent
  VARIANT 3: Xaa is N or D
  VARIANT 9: Xaa is S or D
  VARIANT 10: Xaa is H or D
  VARIANT 28: Xaa is any amino acid
  VARIANT 29: Xaa is L-proline, D-proline, D-hydroxyproline or L-hydroxyproline
  VARIANT 30: Xaa is A, T, or absent
  VARIANT 31: Xaa is R, RR, K, KK, RK, KR or is absent
  VARIANT 32: Xaa is only present when Xaa at position 31 is present and is E or all or a portion of EAEDLQVGQVELGGGPGAGSLQPLALEGSLQKR
XXXQHLCGXXLVEALYLVCGERGFFYTXXXXX